(19) United States
(12) Patent Application Publication
(10) Pub. No.: US 2026/0317710 A1
(43) Pub. Date: Oct. 15, 2026

(54) IN VITRO METHOD FOR OBTAINING CLINICAL-GRADE CD8+ CD45RCLOW/- REGULATORY T CELLS

(71) Applicants: NANTES UNIVERSITE, Nantes (FR); INSERM (INSTITUT NATIONAL DE LA SANTÉ ET DE LA RECHERCHE MÉDICALE), Paris (FR)

(72) Inventors: Carole GUILLONNEAU, Nantes (FR); Ignacio ANEGON, Nantes (FR); Séverine BEZIE, Nantes (FR)

(73) Assignees: NANTES UNIVERSITE, Nantes (FR); INSERM (INSTITUT NATIONAL DE LA SANTÉ ET DE LA RECHERCHE MÉDICALE), Paris (FR)

(21) Appl. No.: 19/101,107

(22) PCT Filed: Aug. 4, 2023
(86) PCT No.: PCT/EP2023/071692

§ 371 Date: Feb. 4, 2025

(30) Foreign Application Priority Data
Aug. 4, 2022 (EP) 22306187.0

(51) Int. Cl. C12N 5/0783; A61K 35/17; A61K 40/11; A61K 40/22; A61P 37/06; C12N 5/00

(52) U.S. Cl. CPC C12N 5/0637; A61K 35/17; A61K 40/11; A61K 40/22; A61P 37/06; C12N 5/0087; C12N 2501/2302; C12N 2501/2315; C12N 2501/51; C12N 2501/515; C12N 2510/00

(57) ABSTRACT

An in vitro method for obtaining clinical-grade CD8⁺ CD45RC^{low/-} regulatory T (Treg) cells, to a pharmaceutical composition including this population of Treg cells. Also, the use of pharmaceutical composition for the prevention or treatment of transplant rejection, graft-versus-host-disease (GVHD), autoimmune disease, chronic inflammatory disease or allergy.

### FIELD OF INVENTION

The present invention relates to an in vitro method for obtaining clinical-grade CD8⁺ CD45RC^{low/-} regulatory T (Treg) cells, to a pharmaceutical composition comprising said population of Treg cells and uses thereof.

### BACKGROUND OF INVENTION

Regulatory T cells, or “Treg” cells, which encompass CD4+ and CD8+ Foxp3+ Treg cells and CD45RC^{low/-} Treg cells are fundamental in controlling various immune responses in that Treg cells can rapidly suppress the activity of other immune cells. In particular, Treg cells are crucial for maintaining tolerance by downregulating undesired immune responses to self and non-self antigens. For instance, Treg defects have been discovered in patients with multiple sclerosis (MS), type I diabetes (T1D), psoriasis, myasthenia gravis (MG) and other autoimmune diseases. Similar links may also exist for atopy and allergic diseases. For all these diseases reports exist pointing to a reduced in vitro immune suppression of the patient's Treg cells. This has led to an increasing interest in the possibility of using Tregs in immunotherapy to treat or prevent autoimmune diseases, allergies and transplantation-related complications, such as graft rejection or graft-versus-host disease (GvHD).

For instance, organ transplantation has seen very significant improvements in both the prevention and treatment of acute rejection, but subclinical episodes and chronic graft dysfunction still heavily impact medium and long-term graft survival. Emerging therapeutic strategies, among them tolerance induction to donor antigens, are moving to the clinical stage after years of experimental model work. Among natural mechanisms and tolerance inductive strategies, the use of different types of regulatory cells, including CD8+ regulatory T cells (CD8+ Tregs), has been recently proposed in the transplantation field, as well as in other pathological situations.

Hence, there is a particular need for human clinical-grade Treg cells, in particular human clinical-grade CD8⁺ CD45RC^{low/-} Treg cells, having a high degree of purity, that are suitable for clinical use, for instance in the fields of transplantation, GVHD autoimmunity, chronic inflammatory diseases or allergy, to avoid degradation of self or therapeutic molecules/tissues by the immune system.

Therefore, there is a need for an in vitro method:
allowing to produce a high number of human clinical-grade Treg cells,
in a reasonable period of time,
said clinical-grade Treg cells having immunosuppressive capacity, and
said clinical-grade Treg cells being suitable and safe enough for clinical use in humans.

### SUMMARY

The invention relates to an in vitro method for obtaining clinical-grade CD8⁺ CD45RC^{low/-} regulatory T (Treg) cells, comprising the steps of:
(a) isolating CD8⁺ cells from a biological sample containing human peripheral blood mononuclear cells (PBMCs) or lymphocytes,
(b) isolating CD8⁺ CD45RC^{low/-} Treg cells from the cells obtained at step (a) by flow cytometry in a closed system using a clinical-grade anti-CD45RC antibody,
(c) stimulating CD8⁺ CD45RC^{low/-} Treg cells obtained at step (b) with a clinical-grade anti-CD3 antibody and a clinical-grade anti-CD28 antibody,
(d) culturing the stimulated Treg cells obtained at step (c) in a medium selected from the group consisting of X-VIVO™ 15, LymphoONE™, JmmunoCult™-XF, PRIME-XV® and CTS™ OpTmizer™, said medium being supplemented with clinical-grade rapamycin and clinical-grade IL-2.

In some embodiments, step (a) is performed with a clinical-grade anti-CD8 antibody, preferably coated to beads.

In some embodiments, step (b) comprises isolating CD8⁺ CD45RC^{low/-} CD56⁻ Treg cells by further using an anti-CD56 antibody. Step (b) may comprise isolating CD8⁺ CD45RC^{low/-} CD56⁻ Treg cells by eliminating CD56⁺ cells by positive selection on a column.

In some embodiments, step (a) is performed using CliniMACS® Plus System (Miltenyi Biotec) and/or step (b) is performed using a cell sorter selected from the group consisting of MACSQuant® Tyto® Cell Sorter, CGX10 (Sony) and WOLF® Cell Sorter.

In some embodiments, the anti-CD3 antibody and the anti-CD28 antibody are both used at a concentration of between 1 μg/mL and 10 μg/mL, preferably 1 μg/mL.

In some embodiments, the IL-2 concentration at step (d) is of between 25 U/mL and 1000 U/mL, between 25 U/mL and 500 U/mL, between 25 U/mL and 250 U/mL, between 25 U/mL and 100 U/mL, between 25 U/mL and 50 U/mL, preferably of 25 U/mL; and/or wherein the rapamycin concentration at step (d) is of between 25 nM and 100 nM, preferably between 25 nM and 50 nM.

In some embodiments, the medium of step (d) is supplemented with a serum selected from the group consisting of CTS™ Immune cell serum replacement, and human serum, preferably CTS™ Immune cell serum replacement, and/or wherein the medium of step (d) is supplemented with IL-15.

In some embodiments, in step (d), the cells are seeded at at least 1×10⁵ cells/cm² into the recipient of culture at day 0.

In some embodiments, the duration of step (d) is of between 14 days and 31 days, preferably 21 days.

In some embodiments, the in vitro method further comprises:
(e) stimulating the cells obtained at step (d) with a clinical-grade anti-CD3 antibody and a clinical-grade anti-CD28 antibody, preferably after 7 and/or 14 days of culture.

In some embodiments, steps (a) to (d), and optionally (e), are performed in closed systems.

In some embodiments, the clinical-grade CD8⁺ CD45RC^{low/-} regulatory T (Treg) cells obtained by said method are GITR+, CD127− and/or Foxp3+ cells.

The present invention also relates to a pharmaceutical composition comprising clinical-grade CD8⁺ CD45RC^{low/-} regulatory T (Treg) cells obtainable by the in vitro method according to any one of claims 1 to 12.

The present invention further relates to a pharmaceutical composition comprising clinical-grade CD8⁺ CD45RC^{low/-} regulatory T (Treg) cells obtainable by the in vitro method according to any one of claims 1 to 12 for use as a medicament.

The present invention further pertains to a pharmaceutical composition comprising clinical-grade CD8⁺ CD45RC^{low/-} regulatory T (Treg) cells obtainable by the in vitro method according to any one of claims 1 to 12 for use in the prevention or treatment of transplant rejection, graft-versus-host-disease (GVHD), autoimmune disease, chronic inflammatory disease or allergy.

### DETAILED DESCRIPTION

The aim of the inventors was to provide a fully optimized method for obtaining clinical-grade CD8⁺ CD45RC^{low/-} regulatory T (Treg) cells suitable to be used in therapy, in particular in human therapy.

The inventors thus developed a method wherein CD8⁺ CD45RC^{low/-} cells from healthy volunteers can be expanded in GMP-grade culture conditions, while preserving their suppressive activity, and assessed and optimized each parameter of said method including the apparatus, reagents, media, concentrations, culture conditions, etc.

In particular:
they identified the most suitable CD8+ T cell subset for cell therapy,
they explored various methods for the GMP-compatible isolation of CD45RClow/− cells and defined a new sorting strategy using available or custom clinical grade monoclonal antibodies,
they refined the PBMC and CD8+ cells GMP-compatible isolation processes,
they explored several methods of GMP stimulation of the cells,
they identified GMP culture media suitable for CD8+ Tregs culture
they identified GMP chemical supplements that are required for CD8+ Tregs culture, and
they defined ideal cell densities at seeding to launch the culture in GMP medium.

A first aspect of the invention relates to an in vitro method for obtaining clinical-grade CD8⁺ CD45RC^{low/-} regulatory T (Treg) cells, comprising the steps of:
(a) isolating CD8⁺ cells from a biological sample containing human peripheral blood mononuclear cells (PBMCs) or lymphocytes,
(b) isolating CD8⁺ CD45RC^{low/-} Treg cells from the cells obtained at step (a) by flow cytometry in a closed system using a clinical-grade anti-CD45RC antibody, (c) stimulating CD8⁺ CD45RC^{low/-} Treg cells obtained at step (b) with a clinical-grade anti-CD3 antibody and a clinical-grade anti-CD28 antibody,
(d) culturing the stimulated Treg cells obtained at step (c) in a medium selected from the group consisting of X-VIVO™ 15, LymphoONE™, JmmunoCult™-XF, PRIME-XV® and CTS™ OpTmizer™, preferably X-VIVO™ 15, said medium being supplemented with clinical-grade rapamycin at a concentration of between 25 nM and 100 nM and clinical-grade IL-2 at a concentration of between 25 U/mL and 1000 U/mL.

The term “clinical grade” refers to products or materials which are suitable for clinical or therapeutic use, such as e.g. injectable grade. Such products or materials are safe enough for human use. For instance, such products or materials fulfil the requirements corresponding to the standards of:
the European or American Pharmacopoeia
the Good Manufacturing Process (GMP)
the International Council for Harmonization guidelines, and/or
the EMEA guidelines: EMEA/CHMP/410869/2006.

The in vitro method for obtaining clinical-grade CD8⁺ CD45RC^{low/-} regulatory Treg cells comprises a step (a) of isolating CD8⁺ cells from a biological sample containing human PBMCs or lymphocytes.

The biological sample is or has been obtained from a subject. As used herein, the term “subject” is intended to include living organisms in which an immune response can be elicited (e.g., mammals, in particular human, primates, dogs, cats, horses, sheep and the like). In some embodiments, the subject is a human. In some embodiments, a subject may be a “patient”, i.e., a warm-blooded animal, preferably a human, who/which is awaiting the receipt of, or is receiving medical care or was/is/will be the object of a medical procedure or is monitored for the development of the targeted disease or condition, such as, for example, kidney failure or dialysis. In some embodiments, the subject is an adult (for example a subject above the age of 18). In another embodiment, the subject is a child (for example a subject below the age of 18). In some embodiments, the subject is a male. In another embodiment, the subject is a female.

In some embodiments, the biological sample is harvested from a healthy subject. In other embodiment, the biological sample is harvested from a patient having a pathology such as e.g. kidney failure. Said patient may be under dialysis.

As used herein, the term “biological sample” refers to any sample obtained from the subject liable to contain CD8⁺ cells. In some embodiments, the biological sample is a blood sample. In some embodiments, the biological sample is a cerebrospinal fluid sample. In some embodiments, the biological sample is a biopsy sample. In some embodiments, the biological sample is a PBMC sample. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

In some embodiments, the sample comprises lymphocytes, in particular CD8+ T cells, generated from induced pluripotent stem cells (iPS). Alternatively, the sample may comprise lymphocytes, in particular CD8+ T cells, generated from embryonic stem (ES) cells.

In some embodiments, human PBMCs are isolated from the blood of a subject using a Ficoll. Alternatively, human PBMCs may be isolated from the blood of a subject using a SepMate™ PBMC isolation tube.

In some embodiments, human PBMCs are isolated from the blood of a subject using cytapheresis.

In some embodiments, the cells used in the present invention are from a blood sample and excludes cells obtained by destruction of a human embryo.

In order to isolate CD8⁺ CD45^{low/-} Treg cells it is preferred to pre-enriched CD8⁺ cells first. Performing the pre-enrichment of CD8⁺ T cells before isolating the CD8⁺ CD45RC^{low/-} Treg cells allows to reduce the duration of the Treg cells isolation and thus to preserve the quality and the quantity of the Treg cells obtained after isolation.

As used herein, the term “regulatory T cells” or “Tregs”, formerly known as suppressor T cells, refers to a subpopulation of T cells which modulate the immune system, maintain tolerance to self-antigens, and abrogate autoimmune diseases. These cells generally suppress or downregulate induction and proliferation of effector T cells.

As used herein, “isolated” refers to a cell or a cell population that is removed from its natural environment (such as the peripheral blood) and that is isolated, purified or separated, and is at least about 50% free, 60% free, 65% free, 70% free, 75% free, 80% free, 85% free and preferably about 90%, 95%, 96%, 97%, 98%, 99% free, from other cells with which it is naturally present, but which lack the cell surface markers based on which the cells are isolated.

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest and express the markers of interest (e.g. a population of human CD8⁺ CD45RC^{low/-} Treg cells comprises at least about 50%, preferably at least about 60%, more preferably at least about 70%, and even more preferably at least about 80% of cells which have the highly suppressive functions and which express the particular markers of interest).

As used herein, the term “marker” refers to a protein, glycoprotein, or any other molecule expressed on the surface of a cell, and which can be used to help identify the cell. A marker can generally be detected by conventional methods. Specific, non-limiting examples of methods that can be used for the detection of a cell surface marker are immunocytochemistry, fluorescence activated cell sorting (FACS), and enzymatic analysis.

As used herein, the term “CD8” (cluster of differentiation 8) well known in the art refers to a transmembrane glycoprotein that serves as a co-receptor for the T cell receptor (TCR). To function, CD8 forms a dimer, consisting of a pair of CD8 chains. The naturally occurring human CD8-α protein has an amino acid sequence provided in the UniProt database under accession number P01732. The naturally occurring human CD8-β protein has an amino acid sequence provided in the UniProt database under accession number P10966.

The pre-enrichment, isolation and/or quantification of a population expressing a determined cell-surface marker, in particular a population of CD8⁺ cells or of CD8⁺ CD45RC^{low/-} Treg cells, may be carried out by a variety of methods for detecting a particular immune cell population available for a skilled artisan, including immunoselection techniques, such as high-throughput cell sorting using flow cytometric methods, affinity methods with antibodies labeled to magnetic beads, biodegradable beads, non-biodegradable beads, and combination of such methods.

As used herein, the term “flow cytometric methods” refers to a technique for counting cells of interest, by suspending them in a stream of fluid and passing them through an electronic detection apparatus. Flow cytometric methods allow simultaneous multiparametric analysis of the physical and/or chemical parameters of up to thousands of particles per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”.

As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate for instance human CD8+ cells.

Alternatively, isolation of the population of interest can be performed using bead-based sorting methods, such as magnetic beads. Using such methods, cells can be separated and isolated positively or negatively with respect to the particular cell-surface markers. As defined herein, “positive selection” refers to techniques that result in the isolation and detection of cells expressing specific cell-surface markers, while “negative selection” refers techniques that result in the isolation and detection of cells not expressing specific cell-surface markers. In some embodiments, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select human cells of interest.

In some embodiments, the biological sample is contacted with one binding partner or with a panel of binding partners (e.g. antibodies) having specificity for CD3 and/or CD45RC, and positive and negative selection can be then performed for isolating and/or quantifying the population of interest.

The terms “expressing”, “positive”, or “+” and “not expressing”, “negative”, or “−” are well known in the art and refer to the expression level of a cell marker of interest, in that the expression level of the cell marker corresponding to “+” is high or intermediate or low (i.e., the cell marker is expressed or present at the cell surface), and the expression level of the cell marker corresponding to “−” is null (i.e., the cell marker is not expressed, or is absent, at the cell surface).

Means useful for isolating a population of interest are binding partners (such as antibodies) to suitable cell surface molecules or markers. Specific binding partners include capture moieties and label moieties. The capture moieties are those which attach both to the cell, either directly or indirectly, and the product. The label moieties are those which attach to the product and may be directly or indirectly labeled. Specific binding partners include any moiety for which there is a relatively high affinity and specificity between product and its binding partner, and in which the dissociation of the product: partner complex is relatively slow so that the product: partner complex is detected during the labeling or cell separation technique.

The capture moiety may be coupled to the anchoring means (the “anchor moiety”) optionally through a linking moiety, and may also include a linking moiety which multiplies the number of capture moieties available and thus the potential for capture of product, such as branched polymers, including, for example, modified dextran molecules, polyethylene glycol, polypropylene glycol, polyvinyl alcohol, and polyvinylpyrrolidone. When the capture moiety is an antibody, it may be referred to as the “capture antibody” or “catch antibody.” As used herein, the term “antibody” is intended to include polyclonal and monoclonal antibodies, chimeric antibodies, single domains antibodies, haptens and antibody fragments, bispecific antibodies, trispecific antibodies and molecules which are antibody equivalents in that they specifically bind to an epitope on the product antigen.

Typically, the antibodies are labeled. The label moiety that can be conjugated to a binding partner such as an antibody are well known to the skilled in the art. For example, radioisotopes, e.g. 32P, 35S or 3H; fluorescence or luminescence markers, e.g. fluorescein (FITC), rhodamine, texas red, phycoerythrin (PE), allophycocyanin, peridinin-chlorophyll-protein complex (PerCP), 6-carboxyfluorescein (6-FAM), 2′, 7′-dimethoxy-4′, 5′-dichloro-6-carboxyfluorescein (JOE), 6-carboxy-X-rhodamine (ROX), 6-carboxy-2′, 4′, 7′, 4, 7-hexachlorofluorescein (HEX), 5-carboxyfluorescein (5-FAM) or N, N, N′, N′-tetramethyl-6-carboxyrhodamine (TAMRA); antibodies or antibody fragments, e.g. F(ab)2 fragment; affinity labels, e.g. biotin, avidin, agarose, bone morphogenetic protein (BMP), matrix bound, haptens; and enzymes or enzyme substrates, e.g. alkaline phosphatase (AP) and horseradish peroxidase (HRP).

Examples of antibodies which bind the human CD8 antigen that are contemplated by the invention include the monoclonal antibodies C8/144B, SK1, 3B5, OKT8, BW135/80 and RPA-T8.

In some embodiments, PBMCs are stained with clinical grade anti-CD8 antibody. In some embodiments, the clinical-grade anti-CD8 antibody is coated to beads. In some embodiments, the cells stained with the anti-CD8 coated beads are magnetically isolated using a column allowing the positive selection of cells and a magnet, preferably a LS column.

Preferably, step (a) of the method of the invention is performed in a closed system wherein the blood of the subject or patient is directly stained with clinical grade anti-CD8 antibody, preferably coated to beads, and then isolated using a closed system.

In some embodiments, the closed system is the CliniMACS® (Miltenyi Biotec). Using CliniMACS® to obtained CD8⁺ T cells dispense from performing a Ficoll which would lead to a cell loss during the different washing steps.

As used herein, an enriched population of human CD8⁺ T cells is one in which the percentage of human CD8⁺ T cells is higher than the percentage of human CD8⁺ T cells in the originally obtained population of cells. Although possible, an enriched population of human CD8⁺ T cells need not contain a homogenous population of human CD8⁺ T cells. In particular embodiments, at least about 50%, about 55%, about 60%, about 65%, about 70%, about 75%, about 80%, about 85%, about 90%, about 95%, about 98%, or about 99% of said cells of the composition human CD8⁺ T cells. In another embodiment, the percentage of human CD8⁺ T cells in the enriched population is at least twice, 5 times, 10 times, 20 times, 30 times, 40 times, 50 times, 60 times, 70 times, 80 times, 90 times, 100 times the percentage of human CD8⁺ T cells before enrichment.

In some embodiments, the percentage of CD8⁺ cells obtained at the end of step (a) is of at least 50, 55, 60, 65, 70, 75, 80, 85, 90, 95 or 99%, preferably of at least 80%, more preferably of at least 90% among the total cells.

In some embodiments, step (a) of the method of the invention is performed at a temperature of between 18° C. and 24° C., between 20° C. and 22° C., preferably at 21° C.

In some embodiments, the duration of step (a) is of between 2 hour and 6 hours, between 3 hour and 5 hours, preferably of about 4 hours.

In some embodiments, after step (a) of the in vitro method, CD8⁺ CD45RC^{low/-} Treg cells are isolated during step (b).

As used herein, the term “CD45” (also known as LCA or PTPRC) refers to a transmembrane glycoprotein existing in different isoforms. These distinct isoforms of CD45 differ in their extracellular domain structures which arise from alternative splicing of 3 variable exons coding for part of the CD45 extracellular region. The various isoforms of CD45 have different extracellular domains, but have the same transmembrane and cytoplasmic segments having two homologous, highly conserved phosphatase domains of approximately 300 residues. The naturally occurring human CD45 protein has an amino acid sequence provided in the UniProt database under accession number P08575. As used herein, the term “CD45RC” refers to the exon 6 splice variant (exon C) of the tyrosine phosphatase CD45. The CD45RC isoform is expressed on B and T cells, and on subsets of CD4+ and CD8+ T cells.

Examples of antibodies which bind the human CD45RC antigen that are contemplated by the invention include the monoclonal antibodies MT2 and RP1/12.

GITR is a marker expressed by CD45RC^{low/-} Treg cells but not by CD45RC^{high} Treg cells. Therefore, in some embodiments, CD8⁺ CD45RC^{low/-} Treg cells are isolated during step (b) using a clinical-grade anti-GITR antibody.

In some embodiments, the CD8⁺ pre-enriched cells are stained during step (b) with a clinical grade anti-CD45RC antibody to isolate the CD8⁺ CD45RC^{low/-} Treg cells using a cell sorter.

Preferably, step (b) is performed in a closed system. In some embodiments, the closed system is selected from the group comprising the MACSQuant® Tyto® Cell sorter (Miltenyi), the CGX10 (Sony) and the WOLF® Cell Sorter (Nanocellect).

It may be desirable for safety reason that the clinical-grade CD8⁺ CD45RC^{low/-} regulatory T (Treg) cells obtained by the in vitro method of the invention be substantially devoid of MAIT (Mucosal-Associated Invariant T) cells and/or NK (Natural Killer) cells. Therefore, in vitro method for obtaining clinical-grade CD8⁺ CD45RC^{low/-} regulatory Treg cells according to the invention may comprise a step of eliminating, for instance negatively selecting, MAIT cells and/or NK cells. In some embodiments, the in vitro method of the invention comprises a step of eliminating MAIT cells and/or NK cells by negative selection using an anti-CD56 antibody.

In some embodiments, step (b) comprises isolating CD8⁺ CD45RC^{low/-} CD56⁻ Treg cells by negative selection by further using an anti-CD56 antibody. In some embodiments, the anti-CD56 antibody is a clinical grade antibody. In some embodiments, the anti-CD56 antibody is not a clinical grade antibody.

In some embodiments, step (b) comprises isolating CD8⁺ CD45RC^{low/-} CD4⁻ Treg cells by negative selection by further using an anti-CD4 antibody. In some embodiments, the anti-CD4 antibody is a clinical grade antibody. In some embodiments, the anti-CD4 antibody is not a clinical grade antibody.

In some embodiments, the percentage of CD8⁺ CD45RC^{low/-} cells obtained at the end of step (b) is of at least 50, 55, 60, 65, 70, 75, 80, 85, 90, 95 or 99%, preferably of at least 90%, more preferably of at least 95% among the total cells.

In some embodiments, the step (b) is performed at a temperature of between 18° C. and 24° C., between 20° C. and 22° C., preferably at 21° C.

In some embodiments, the duration of step (b) is of between 2 hour and 6 hours, between 3 hour and 5 hours, preferably of 4 hours.

A polyclonal expansion of a Treg cell population of interest may be obtained by using an anti-CD3 antibody and an anti-CD28 antibody.

Therefore, after steps (a) and (b) of the in vitro method, CD8⁺ CD45RC^{low/-} Treg cells are stimulated during step (c) with a clinical-grade anti-CD3 antibody and a clinical-grade anti-CD28 antibody.

In some embodiments, the clinical-grade anti-CD3 and anti-CD28 antibodies are monoclonal antibodies.

In some embodiments, the clinical-grade anti-CD3 is used at a concentration of between 0.5 μg/mL and 20 μg/mL, between 0.5 μg/mL and 10 μg/mL, between 1 μg/mL and 10 μg/mL, between 1 μg/mL and 5 μg/mL, preferably about 1 μg/mL.

In some embodiments, the clinical-grade anti-CD28 is used at a concentration of between 0.5 μg/mL and 20 μg/mL, between 1 μg/mL and 10 μg/mL, between 1 μg/mL and 5 μg/mL, preferably about 1 μg/mL.

In some embodiments, the clinical-grade anti-CD3 and anti-CD28 antibodies are used at a same concentration.

In some embodiments, anti-CD3 and anti-CD28 antibodies are both used at a concentration of between 1 μg/mL and 10 μg/mL, between 1 μg/mL and 5 μg/mL, preferably about 1 μg/mL.

Preferably, the clinical-grade anti-CD3 antibody is not coated on beads. In some embodiments, the clinical-grade anti-CD3 antibody is coated on the culture support.

Preferably, the clinical-grade anti-CD28 antibody is not coated on beads. In some embodiments, the clinical-grade anti-CD28 antibody is soluble in the culture.

In embodiments wherein the clinical-grade anti-CD3 and anti-CD28 antibodies are not coated on beads, no extra wash is required to eliminate the beads present in the culture medium, which avoid cell loss.

In some embodiments, the CD8⁺ CD45RC^{low/-} Treg cells are stimulated with clinical-grade anti-CD3 and anti-CD28 antibodies at at least day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 and/or 31 of culture.

In some embodiments, the CD8⁺ CD45RC^{low/-} Treg cells are stimulated with anti-CD3 and anti-CD28 antibodies at day 0. Anti-CD3 antibody is coated in the culture support at every harvesting and seeding of Treg cells. In some embodiments, the cells may be harvested and seeded in a new support of culture every 2, 3, 4, 5, 6, or 7 days, preferably 7 days. In some embodiments, in every step of harvest and seed in a new support of culture, clinical-grade anti-CD28 is added in the culture medium and clinical-grade anti-CD3 is coated in the new support. In some embodiments, clinical-grade anti-CD28 antibody is added in the culture medium every 2, 3, 4, 5, 6, or 7 days, preferably 7 days. In some embodiments, at day 0, 7, 14 and 21 of culture, CD8⁺ CD45RC^{low/-} Treg cells are stimulated with anti-CD3 and anti-CD28 antibodies. In some embodiments, at day 0, 7, 14 and 21 of culture, clinical-grade anti-CD28 is added in the culture media and anti-CD3 is coated in the new support.

In some embodiments, step (c) occurs at the beginning of step (d) and continues during step (d), i.e. the cells are repeatedly stimulated during the step of culture.

Preferably, step (c) of the method of the invention is performed in a closed system. In some embodiments, the closed system is selected from the group comprising or consisting of flasks, a petri dishes, culture plates from 6 to 24 wells, stirrer, bottles, cell bags (CultiLife from Takara, GMP cell expansion bags from Miltenyi), CentriCult Chamber (Miltenyi Biotec associated with the Prodigy system), G-Rex Cell culture devices (WilsonWolf).

In some embodiments, step (c) is performed at a temperature of about 37° C.

As used herein, the term “expanding” refers to the process of converting and/or amplifying a given population of cells (e.g. immune cells such as T cells). Expansion of T cells is preferably performed by culturing a cell population comprising T cells in the presence of antigen-specific stimulating agent such as, for example, antigens, cells, antibodies, lectins, etc. Expansion may also require culture of T cells in the presence of one or more cytokine(s).

In some embodiments, after steps (a), (b) and (c) of the in vitro method, stimulated CD8⁺ CD45RC^{low/-} Treg cells are cultured in step (d) in a clinical-grade medium.

As used herein, the term “medium” refers to a medium for maintaining a cell population, or culturing a cell population (e.g. “culture medium”) containing nutrients that maintain cell viability and support proliferation. The medium may contain any of the following in an appropriate combination: salt(s), buffer(s), amino acids, glucose or other sugar(s), antibiotics, serum or serum replacement, and other components such as growth factors, cytokines etc. Media ordinarily used for particular cell types are known to those skilled in the art. The medium of the invention may be based on a commercially available medium.

The inventors tested and compared the efficacy of various media including Biotarget, TexMACS, X-VIVO15, CTS™ AIM V, CTS™ OpTmizer™ LymphoONE™, PRIME-XV, SCGM and ImmunoCult™-XF. They compared the expansion and suppression score of the cells obtained after culture in each of these media to those of cells obtained after culture in RPMI1640 research grade medium (used as a reference).

Thus, in some embodiments, the clinical-grade medium is selected from the group comprising or consisting of Biotarget, TexMACS, X-VIVO15, CTS™ AIM V, CTS™ OpTmizer™, LymphoONE™, PRIME-XV, SCGM and ImmunoCult™-XF.

Preferably, the clinical-grade medium is selected from the group comprising or consisting of X-VIVO™ 15, LymphoONE™, ImmunoCult™-XF, PRIME-XV® and CTS™ OpTmizer™.

In some embodiments, the clinical-grade medium is X-VIVO™ 15.

In some embodiments, the culture medium also comprises at least one clinical-grade cytokine including human IL-2.

As used herein the terms “Interleukin-2” or “IL-2” are well known in the art and refer to cytokine which is important for the proliferation of T and B lymphocytes. The naturally occurring human IL-2 protein has an aminoacid sequence of 153 amino acids provided in the UniProt database under accession number P60568.

In some embodiments, IL-2 is a human interleukin-2 (hIL-2), preferably a recombinant human interleukin-2 (rhIL-2). rhIL-2 is commercially available for pharmaceutical uses. Suitable commercial forms include, e.g. Proleukin®, a recombinant human IL-2 composition.

In some embodiments, the culture medium further comprises at least another clinical-grade cytokine, such as e.g. human IL-15.

In some embodiments, the at least one clinical-grade cytokine, for instance IL-2 and/or IL-15, is added in the culture medium at at least day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of culture. In some embodiments, the at least one clinical-grade cytokine is added every two days of culture. In some embodiments, the at least one clinical-grade cytokine is added at days 0, 2, 4, 6, 8, 10, 12, 14, 16, 18, 20, 22, 24, 26, 28 and 30 of culture.

In some embodiments, the at least one cytokine added to the culture medium is hIL-2. In some embodiments, hIL-2 and hIL-15 are added to the culture medium.

In some embodiments, the hIL-2 concentration at step (d) is of between 25 U/mL and 1000 U/mL, between 25 U/mL and 500 U/mL, between 25 U/mL and 250 U/mL, between 25 U/mL and 100 U/mL, between 25 U/mL and 50 U/mL, preferably of about 25 U/mL.

In some embodiments, the hIL-2 concentration at step (d) is of between 2 U/mL and 1000 U/mL, between 5 U/mL and 1000 U/mL, between 10 U/mL and 1000 U/mL, between 15 U/mL and 1000 U/mL, between 20 U/mL and 1000 U/mL, between 25 U/mL and 1000 U/mL.

In some embodiments, the hIL-2 concentration at step (d) is of between 2 U/mL and 50 U/mL, between 5 U/mL and 50 U/mL, between 10 U/mL and 50 U/mL, between 15 U/mL and 50 U/mL, between 20 U/mL and 50 U/mL, between 25 U/mL and 50 U/mL.

In some embodiments, the clinical-grade cytokine hIL-2 is added in the culture medium at step (d) at a concentration of between 2 nM and 1000 nM, between 5 nM and 1000 nM, between 10 nM and 1000 nM, between 15 nM and 1000 nM, between 20 nM and 1000 nM, between 25 nM and 1000 nM.

In some embodiments, the clinical-grade cytokine hIL-2 is added in the culture medium at step (d) at a concentration of between 2 nM and 100 nM, between 5 nM and 100 nM, between 10 nM and 100 nM, between 15 nM and 100 nM, between 20 nM and 100 nM, between 25 nM and 100 nM.

In some embodiments, the clinical-grade cytokine hIL-15 is added in the culture medium at a concentration of between 10 ng/mL and 500 ng/mL, between 10 ng/mL and 250 ng/mL, between 10 ng/mL and 100 ng/mL, between 10 ng/mL and 50 ng/mL and preferably 10 ng/mL.

Other cytokines, such as e.g. hIL-10 and/or hTGF-β, may be further added to the culture medium of step (d). In some embodiments, hIL-10 and/or hTGF-β are added to the culture medium of step (d) at a concentration of between 2 ng/mL and 100 ng/mL, between 2 ng/mL and 50 ng/mL, preferably 5 ng/mL.

As used herein, the term “rapamycin” includes compounds having the rapamycin core structure as defined in U.S. Patent Application Publication No. 2003/0008923 (which is herein incorporated by reference), which may be chemically or biologically modified while still retaining mTOR inhibiting properties. In some embodiments, the rapamycin compound is rapamycin. The derivatives include esters, ethers, oximes, hydrazones, and hydroxylamines of rapamycin, as well as compounds in which functional groups on the rapamycin core structure have been modified, for example, by reduction or oxidation. Pharmaceutically acceptable salts of such compounds are also considered to be rapamycin derivatives. Specific examples of esters and ethers of rapamycin are esters and ethers of the hydroxyl groups at the 42- and/or 31-positions of the rapamycin nucleus, and esters and ethers of a hydroxyl group at the 27-position (following chemical reduction of the 27-ketone). Specific examples of oximes, hydrazones, and hydroxylamines are of a ketone at the 42-position (following oxidation of the 42-hydroxyl group) and of 27-ketone of the rapamycin nucleus.

Other compounds within the scope of “rapamycin analog or derivative thereof” include those compounds and classes of compounds referred to as “rapalogs” and “epirapalogs”. Another compound within the scope of “rapamycin derivatives” is everolimus, a 4-O-(2-hydroxyethyl)-rapamycin derived from a macrolide antibiotic produced by Streptomyces hygroscopicus (Novartis). Everolimus is also known as Certican, RAD-001 and SDZ-RAD.

In some embodiments, the rapamycin is added in the culture medium at at least day 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 31 of culture. In some embodiments, the rapamycin is added every two days of culture. In some embodiments, the rapamycin is added at days 2, 4, 6, 8, 10, 12, 14, 16, 18, 20, 22, 24, 26, 28 and 30 of culture.

In some embodiments, clinical-grade rapamycin is added to the culture medium of step (d) at a concentration of between 25 nM and 500 nM, between 25 nM and 250 nM, between 25 nM and 100 nM, between 25 and 50 nM, preferably about 50 nM.

In some embodiments, clinical-grade rapamycin is added to the culture medium at step (d) at a concentration of between 2 nM and 500 nM, between 5 nM and 500 nM, between 10 nM and 500 nM, between 15 nM and 500 nM, between 20 nM and 500 nM, between 25 nM and 500 nM.

In some embodiments, clinical-grade rapamycin is added to the culture medium at step (d) at a concentration of between 2 nM and 100 nM, between 5 nM and 100 nM, between 10 nM and 100 nM, between 15 nM and 100 nM, between 20 nM and 100 nM, between 25 nM and 100 nM.

In some embodiments, further to rapamycin, clinical-grade cyclosporine A, mycophenolate mofetil, methylprednisolone, tacrolimus or combinations thereof may be added to the culture medium at step (d).

In some embodiments, clinical-grade cyclosporine A is added to the culture medium at step (d) at a concentration of between 1 ng/mL and 100 ng/mL. In some embodiments, clinical-grade mycophenolate mofetil is added to the culture medium at step (d) at a concentration of between 0.1 and 5 μg/mL. In some embodiments, clinical-grade methylprednisolone is added to the culture medium at step (d) at a concentration of between 10 and 1000 pg/mL. In some embodiments, clinical-grade tacrolimus is added to the culture medium at step (d) at a concentration of between 0.1 and 5 pg/mL.

It may be desirable to avoid using human serum in the cell culture medium, notably for safety reasons. Therefore, the inventors tested various cell culture conditions in the absence of serum in order to identify optimized culture media allowing high expansion of the cells of interest, preferably in the absence of serum, in particular of human serum.

Therefore, in some embodiments, the medium of step (d) is not supplemented with serum.

However, the culture medium may also be supplemented with serum.

In some embodiments, the medium of step (d) is supplemented with serum. In some embodiments, the serum is CTS™ Immune cell serum replacement or human serum. Human serum may for instance be human AB serum, i.e. serum from a human donor with the AB blood group. Preferably, the serum is CTS™ Immune cell serum replacement.

In some embodiments, the serum is used at a concentration of at least 1, 2, 3, 4, 5, 6, 7, 8, 9, 10, 11, 12, 13, 14 or 15%.

For instance, CTS™ Immune cell serum replacement may be used at a concentration of about 5%, and human AB serum may be used at a concentration of about 10%.

In some embodiments, the serum is present in the culture medium during the all duration of step (d).

In some embodiments, the medium of step (d) is supplemented with a serum selected from the group comprising CTS™ Immune cell serum replacement and human serum, and/or with hIL-15.

In some embodiments, the medium of step (d) is supplemented with a serum selected from the group comprising CTS™ Immune cell serum replacement and human serum, and with hIL-15.

In some embodiments, the medium of step (d) is not supplemented with serum, and is supplemented with hIL-15.

In some embodiments, step (d) is performed in a closed system. In some embodiments, the closed system is selected from the group comprising or consisting of flasks, culture plates from 6 to 24 wells, cell and bags (CultiLife from Takara, GMP cell expansion bags from Miltenyi), CentriCult Chamber (Miltenyi Biotec associated with the Prodigy system), G-Rex Cell culture devices (WilsonWolf).

In some embodiments, the step (d) is performed at a temperature of 37° C.

In some embodiments, at step (d) the cells are seeded at at least 1×10⁵ cells/cm², 2×10⁵ cells/cm², 4×10⁵ cells/cm², 6×10⁵ cells/cm², 8×10⁵ cells/cm², 1×10⁶ cells/cm², or 2×10⁶ cells/cm², into the recipient of culture at day 0.

Preferably, at step (d) the cells are seeded into the recipient of culture at day 0 at at least 1×10⁵ cells/cm².

In some embodiments, the duration of step (d) is of at least 7, 8, 9, 10, 11, 12, 13, 14, 15, 16, 17, 18, 19, 20, 21, 22, 23, 24, 25, 26, 27, 28, 29, 30 or 31 days. In some embodiments, the duration of step (d) is of between 7 days and 31 days, between 14 days and 31 days, between 14 days and 21 days, preferably 21 days.

In some embodiments, the in vitro method for obtaining clinical-grade CD8⁺ CD45RC^{low/-} Treg cells further comprises a step (e) of stimulating the cells obtained at step (d) with a clinical-grade anti-CD3 antibody and a clinical-grade anti-CD28 antibody.

Preferably, said step (e) of stimulating the cells obtained at step (d) with a clinical-grade anti-CD3 antibody and a clinical-grade anti-CD28 antibody is done after 7, 8, 9, 10, 11, 12, 13, 14, 15, 16, 17, 18, 19, 20 and/or 21 days of culture, preferably after 7 and/or 14 days of culture.

Preferably, the steps (a) to (d) and optionally (e) are performed in closed systems.

In some embodiments, the invention also relates to the clinical-grade CD8⁺ CD45RC^{low/-} Treg cells or cell population obtained or obtainable by the method of the invention.

In some embodiments, the enriched population of Treg contains a percentage of human Treg cells that is at least twice the percentage of human Treg cells within the population before enrichment. In some embodiments, the enriched population of Treg contains a percentage of human Treg cells that is at least 2, 3, 4, 5, 6, 7, 8, 9 or 10-fold the percentage of human Treg cells within the population before enrichment.

In some embodiments, at least 80, 85, 90, 95 or 99% of the clinical-grade cells obtained by the method of the invention are CD8⁺ CD4⁻ CD45RC^{low/-} Treg cells.

In some embodiments, at least 50, 55, 60, 65, 70, 75, 80, 85, 90, 95% of the clinical-grade CD8⁺ CD45RC^{low/-} Treg cells obtained by the method of the invention are Foxp3⁺ and/or GITR⁺ and/or HLA-DR⁺ and/or TGF-β⁺ and/or IL-34⁺ and/or IFN-γ⁺. In some embodiments, at least 50, 55, 60, 65, 70, 75, 80, 85, 90, 95% of the clinical-grade CD8⁺ CD45RC^{low/-} Treg cells obtained by the method of the invention are CD27⁻ and/or CCR7⁻ and/or PD1⁻ and/or CD154⁻ and/or CD127⁻ and/or CD45RA⁻ and/or IL-10. In some embodiments, at least 50, 55, 60, 65, 70, 75, 80, 85, 90, 95% of the clinical-grade CD8⁺ CD45RC^{low/-} Treg cells obtained by the method of the invention are GITR⁺ and/or CD127⁻ and/or Foxp3⁺ cells.

In some embodiments, the invention also relates to clinical-grade CD8⁺ CD45RC^{low/-} Treg cells, or to a population of clinical-grade CD8⁺ CD45RC^{low/-} Treg cells, obtainable by the method of the invention.

The CD8⁺ CD45RC^{low/-} Treg cells obtained or obtainable by the method of the invention present various advantages. In particular, they have one or more of the following properties:
they are clinical-grade human cells that are suitable and safe enough for clinical use in humans,
they have high expansion capacity, allowing to produce a high number of clinical-grade human cells,
they have high immunosuppressive capacity,
they do not display cytotoxic activity against human primary aortic endothelial cells,
they do not display cytotoxic activity against human peripheral blood mononuclear cells (PBMCs),
they contain differentially expressed genes related to cell activation and memory status, or associated with Treg cell immunosuppressive function, such as e.g. inflammation or tolerance compared to cells freshly isolated from peripheral blood, and
they may be genetically modified by lentiviral vectors or using the CRISPR-Cas system.

In some embodiments, the invention also relates to a composition comprising, consisting essentially of or consisting of the clinical-grade CD8⁺ CD45RC^{low/-} Treg cells or cell population obtained or obtainable by the in vitro method of the invention as disclosed hereinabove.

As used herein, the term “consisting essentially of”, with reference to a composition or medicament, means that the clinical-grade CD8⁺ CD45RC^{low/-} Treg cells or cell population obtained or obtainable by the in vitro method of the invention is the only one therapeutic agent or agent with a biologic activity within said composition or medicament.

In some embodiments, the composition is a pharmaceutical composition which further comprises at least one pharmaceutically acceptable excipient.

The term “pharmaceutically acceptable excipient” includes any and all solvents, dispersion media, coatings, antibacterial and antifungal agents, isotonic and absorption delaying agents and the like. Said excipient does not produce an adverse, allergic or other untoward reaction when administered to an animal, preferably a human. For human administration, preparations should meet sterility, pyrogenicity, and general safety and purity standards as required by regulatory offices, such as, for example, FDA Office or EMA.

Pharmaceutically acceptable excipients that may be used in the pharmaceutical composition of the invention include, but are not limited to, ion exchangers, alumina, aluminum stearate, lecithin, serum proteins, such as, for example, human serum albumin, buffer substances such as, for example, phosphates, glycine, sorbic acid, potassium sorbate, partial glyceride mixtures of saturated vegetable fatty acids, water, salts or electrolytes, such as, for example, protamine sulfate, disodium hydrogen phosphate, potassium hydrogen phosphate, sodium chloride, zinc salts, colloidal silica, magnesium trisilicate, polyvinyl pyrrolidone, cellulose-based substances (5 for example sodium carboxymethylcellulose), polyethylene glycol, polyacrylates, waxes, polyethylenepolyoxypropylene-block polymers, polyethylene glycol and wool fat.

In some embodiments, the pharmaceutical compositions according to the present invention comprise vehicles which are pharmaceutically acceptable for a formulation capable of being injected to a subject. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The prevention against contamination by microorganisms can be brought about by adding in the composition preservatives such as, for example, various antibacterial and antifungal agents (for example, parabens, chlorobutanol, phenol, sorbic acid, thimerosal and the like). In an embodiment, it may be preferable to include isotonic agents, for example, sugars or sodium chloride, to reduce pain during injection. In some embodiments, prolonged absorption of the injectable compositions can be brought about by the use in the compositions of agents delaying absorption, for example, aluminum monostearate and gelatin.

In some embodiments, the clinical-grade CD8⁺ CD45RC^{low/-} Treg cells or cell population, the composition, the pharmaceutical composition, or the medicament of the invention is in an adapted form for an injection. Thus, in some embodiments, the cell population, the composition, pharmaceutical composition, or medicament of the invention is to be injected (or is for injection) to the subject by intravenous, intramuscular, intraperitoneal, or subcutaneous injection. Examples of forms suitable for injectable use include, but are not limited to, sterile solutions or dispersions and sterile powders for the extemporaneous preparation of sterile injectable solutions or dispersions.

The invention also provides a medicament comprising a population of clinical-grade CD8⁺ CD45RC^{low/-} Treg cells or cell population obtained or obtainable by the in vitro method of the invention.

As mentioned above, the population of CD8⁺ CD45RC^{low/-} Treg cells of the invention, is of interest in the fields of autoimmunity, chronic inflammation, allergy, transplantation (either for preventing or treating graft rejection or GVHD), gene therapy and treatment with therapeutic protein, to avoid degradation of self-tissues or therapeutic proteins by the immune system. A population of human CD8⁺ CD45RC^{low/-} Treg cells according to the invention indeed exhibits the ability to induce immune tolerance and/or to suppress and/or inhibit immune responses, preferably antigen-specific immune response(s) directed against the antigen(s) involved in the disease to be treated, and/or unwanted adaptive immune responses mediated by CD4+ T cells, CD8+ T cells, preferably immune T-cell tolerance or reduced T-cell activation. Preferably, the population of CD8⁺ CD45RC^{low/-} Treg cells of the invention, is isolated from the patient to be treated (and will be re-administered to the patient after ex vivo expansion).

Alternatively, the population of CD8⁺ CD45RC^{low/-} Treg cells of the invention, may be isolated from a donor and will be administered to a recipient patient after ex vivo expansion. In this case, the population of CD8⁺ CD45RC^{low/-} Treg cells is allogeneic vis-a-vis of the recipient patient to be treated.

In some embodiments, the population of CD8⁺ CD45RC^{low/-} Treg cells of the invention may be genetically modified, for instance in order to express a chimeric antigen receptor (CAR), or to bear a gene deletion or inactivation or a knock-out mutation.

Therefore, another aspect of the invention relates to a population of CD8⁺ CD45RC^{low/-} Treg cells of the invention, or a composition or pharmaceutical composition of the present invention for use as a medicament. Consequently, the present invention further relates to a medicament comprising, consisting essentially of or consisting of the clinical-grade CD8⁺ CD45RC^{low/-} Treg cells or cell population obtained or obtainable by the method of the invention as described above.

The invention also relates to a method for treating a subject in need thereof, comprising administering to the subject a population of clinical-grade CD8⁺ CD45RC^{low/-} regulatory T (Treg) cells, or a pharmaceutical composition comprising clinical-grade CD8⁺ CD45RC^{low/-} regulatory T (Treg) cells, obtained or obtainable by the in vitro method according to the invention.

In some embodiments, the clinical-grade CD8⁺ CD45RC^{low/-} Treg cells or cell population obtained or obtainable by the method of the invention, the composition, or pharmaceutical composition of the invention are administered (or are to be administered) to a subject in a therapeutically effective amount.

The term “therapeutically effective amount” refers to the amount of a composition, pharmaceutical composition, or a number of cells, effective to achieve a particular biological result. A therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.

As used herein, the term “therapeutically effective amount” may also refer to the number of CD8⁺ CD45RC^{low/-} Treg cells of the invention, that is aimed at, without causing significant negative or adverse side effects to the target, (1) delaying or preventing the onset of an unwanted immune response; (2) slowing down or stopping the progression, aggravation, or deterioration of one or more symptoms of an unwanted immune response; (3) bringing about ameliorations of the symptoms of an unwanted immune response; (4) reducing the severity or incidence of an unwanted immune response; or (5) curing an unwanted immune response.

The effective amount will vary with the age, gender, race, general condition, etc., of the patient, the severity of the condition being treated, the duration of the treatment, the nature of any concurrent treatment, the pharmaceutically acceptable carrier used, and like factors within the knowledge and expertise of those skilled in the art.

As used herein, the term “patient” refers to a human, who is awaiting the receipt of, or is receiving, medical care or was/is/will be the subject of a medical procedure, or is monitored for the development or progression of a disease. In some embodiments, the patient is an adult (for example a patient above the age of 18). In another embodiment, the patient is a child (for example a patient below the age of 18). In some embodiments, the patient is a male. In other embodiments, the patient is a female.

The terms “prevent,” “preventing,” and “prevention” refer herein to the inhibition of the development or onset of a disorder or the prevention of the recurrence, onset, or development of one or more symptoms of a disorder in a subject resulting from the administration of the CD8⁺ CD45RC^{low/-} Treg cells of the invention.

As used herein, the term “treatment” refers to therapeutic treatment and prophylactic and preventive measures, wherein the object is to prevent or slow down (lessen, diminish) the targeted pathological disorder or condition. Those in need of treatment include those already with the disorder as well as those prone to have the disorder. A patient is successfully “treated” for an unwanted immune response if, after receiving a therapeutic amount of CD8⁺ CD45RC^{low/-} Treg cells of the invention, the patient shows observable and/or measurable reduction in or absence of one or more of the following: reduction in the number of pathogenic cells; reduction in the percent of total cells that are pathogenic; and/or relief to some extent, of one or more of the symptoms associated with the specific disease or condition; reduced morbidity and mortality, and improvement in quality of life issues. The above parameters for assessing successful treatment and improvement in the disease are readily measurable by routine procedures familiar to a physician.

The clinical-grade CD8⁺ CD45RC^{low/-} Treg cells or cell population, the composition, the pharmaceutical composition, or the medicament of the invention, may be (re)introduced to the patient by a number of approaches. Preferably, they are injected intravenously. In some embodiments, about 1×10⁴ to about 1×10⁸ cells/kg are (re)introduced in the patient. In another embodiment, about 1×10⁶ to about 10×10⁶ cells/kg of patient body weight are (re)introduced to the patient. Preferably, the population of CD8⁺ CD45RC^{low/-} Treg cells of the invention, has been isolated from the patient to be treated and is (re)administered to said patient after ex vivo expansion.

Another aspect of the invention is a population of clinical-grade CD8⁺ CD45RC^{low/-} regulatory T (Treg) cells, or a pharmaceutical composition comprising clinical-grade CD8⁺ CD45RC^{low/-} regulatory T (Treg) cells, obtained or obtainable by the in vitro method according to the invention, for use in the prevention or treatment of transplant rejection, graft-versus-host-disease (GVHD), autoimmune disease, chronic inflammatory disease or allergy.

A further aspect of the invention is a method for preventing and/or treating transplant rejection, graft-versus-host-disease (GVHD), autoimmune disease, chronic inflammatory disease or allergy in a subject in need thereof, comprising administering to the subject a population of clinical-grade CD8⁺ CD45RC^{low/-} regulatory T (Treg) cells, or a pharmaceutical composition comprising clinical-grade CD8⁺ CD45RC^{low/-} regulatory T (Treg) cells, obtained or obtainable by the in vitro method according to the invention.

The present invention further relates to clinical-grade CD8⁺ CD45RC^{low/-} Treg cells or cell population of the invention, or to a composition, a pharmaceutical composition, or a medicament of the invention, for use in the prevention or treatment of transplant rejection.

The present invention thus further relates to a method for preventing, reducing or treating transplant rejection (or for inducing transplant tolerance) in a subject in need thereof, comprising administering to the subject the clinical-grade CD8⁺ CD45RC^{low/-} Treg cells or cell population of the invention, or the composition, pharmaceutical composition, or medicament of the invention.

The terms “preventing transplant rejections” and “reducing transplant rejections” are meant to encompass prevention or inhibition of immune transplant rejection, as well as delaying the onset or the progression of immune transplant rejection. The terms are also meant to encompass prolonging survival of a transplant in a subject, or reversing failure of a transplant in a subject. Further, the terms are meant to encompass ameliorating a symptom of an immune transplant rejection, including, for example, ameliorating an immunological complication associated with immune rejection, such as, e.g., interstitial fibrosis, chronic graft arteriosclerosis, or vasculitis.

The term “transplantation” and variations thereof refer to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an isograft, derived from a human of the same or different genetic origins. In another scenario, the graft is derived from a species different from that into which it is transplanted, including animals from phylogenically widely separated species, for example, a baboon heart being transplanted into a human host.

The term “transplant rejection”, as used herein, encompasses both acute and chronic transplant rejection. “Acute rejection” is the rejection by the immune system of a tissue transplant recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, cyclosporine, anti-CD40L monoclonal antibody and the like. “Chronic transplant rejection” generally occurs in humans within several months to years after engraftment, even in the presence of successful immunosuppression of acute rejection. Fibrosis is a common factor in chronic rejection of all types of organ transplants.

The present invention further relates to clinical-grade CD8⁺ CD45RC^{low/-} Treg cells or cell population of the invention, or to the composition, pharmaceutical composition, or medicament of the invention, for use in the prevention or treatment of graft-versus-host-disease (GVHD).

The present invention further relates to a method for preventing, reducing or treating graft-versus-host-disease (GVHD) (or for inducing transplant tolerance) in a subject in need thereof, comprising administering to the subject the clinical-grade CD8⁺ CD45RC^{low/-} Treg cells or cell population of the invention, composition, pharmaceutical composition or medicament of the invention.

In some embodiments, the donor of the transplant is a human. The donor of the transplant can be a living donor or a deceased donor, namely a cadaveric donor.

In some embodiments, the transplant is an organ, a tissue, or cells.

In certain embodiments the cells are selected from the group consisting of multipotent hematopoietic stem cells derived from bone marrow, peripheral blood, or umbilical cord blood; or pluripotent (i.e. embryonic stem cells (ES) or induced pluripotent stem cells (iPS)) or multipotent stem cell-derived differentiated cells of different cell lineages such as cardiomyocytes, beta-pancreatic cells, hepatocytes, neurons, etc.

In some embodiments, the cell composition is used for allogeneic hematopoietic stem cell transplantation (HSCT) and thus comprises multipotent hematopoietic stem cells, usually derived from bone marrow, peripheral blood, or umbilical cord blood.

HSCT can be curative for patients with leukemia and lymphomas. However, an important limitation of allogeneic HCT is the development of graft versus host disease (GVHD), which occurs in a severe form in about 30-50% of humans who receive this therapy.

In some embodiments, the invention relates to clinical-grade CD8⁺ CD45RC^{low/-} Treg cells or cell population of the invention, or to the composition, pharmaceutical composition, or medicament of the invention for use in the prevention or treatment of an autoimmune disease.

The invention also relates to a method for preventing or treating an autoimmune disease in a subject in need thereof, comprising administering to said subject clinical-grade CD8⁺ CD45RC^{low/-} Treg cells or cell population of the invention, or the composition, pharmaceutical composition, or medicament of the invention.

As used herein, the term “autoimmune disease” refers to a disease in which the immune system produces an immune response (for example, a B-cell or a T-cell response) against an antigen that is part of the normal host (that is an auto-antigen), with consequent injury to tissues. In an autoimmune disease, the immune system of the host fails to recognize a particular antigen as “self” and an immune reaction is mounted against the host's tissues expressing the antigen.

Exemplary autoimmune diseases affecting humans include rheumatoid arthritis, juvenile oligoarthritis, collagen-induced arthritis, adjuvant-induced arthritis, uveitis, Sjogren's syndrome, multiple sclerosis, experimental autoimmune encephalomyelitis, inflammatory bowel disease (for example, Crohn's disease and ulcerative colitis), autoimmune gastric atrophy, pemphigus vulgaris, psoriasis, vitiligo, type 1 diabetes, non-obese diabetes, myasthenia gravis, Grave's disease, Hashimoto's thyroiditis, sclerosing cholangitis, sclerosing sialadenitis, systemic lupus erythematosis, autoimmune thrombocytopenia purpura, Goodpasture's syndrome, Addison's disease, systemic sclerosis, polymyositis, dermatomyositis, acquired hemophilia, thrombotic thrombocytopenic purpura, IPEX, APECED syndrome, Duchenne muscular dystrophy, immunological infertility, autoimmune genetic diseases and the like.

In other embodiments, the invention relates to clinical-grade CD8⁺ CD45RC^{low/-} Treg cells or cell population of the invention, or to the composition, pharmaceutical composition, or medicament of the invention for use in the prevention or treatment of a chronic inflammatory disease.

The invention also relates to a method for preventing or treating a chronic inflammatory disease in a subject in need thereof, comprising administering to said subject clinical-grade CD8⁺ CD45RC^{low/-} Treg cells or cell population of the invention, or the composition, pharmaceutical composition, or medicament of the invention.

Examples of chronic inflammatory diseases affecting humans include, without being limited to, non-autoimmune inflammatory bowel disease, post-surgical adhesions, coronary artery disease, hepatic fibrosis, acute respiratory distress syndrome, acute inflammatory pancreatitis, endoscopic retrograde cholangiopancreatography-induced pancreatitis, burns, atherogenesis of coronary, cerebral and peripheral arteries, appendicitis, cholecystitis, diverticulitis, visceral fibrotic disorders, wound healing, skin scarring disorders (keloids, hidradenitis suppurativa), granulomatous disorders (sarcoidosis, primary biliary cirrhosis), asthma, pyoderma gandrenosum, Sweet's syndrome, Behcet's disease, primary sclerosing cholangitis, and an abscess.

In another embodiment, the invention relates to clinical-grade CD8⁺ CD45RC^{low/-} Treg cells or cell population of the invention, or to the composition, pharmaceutical composition, or medicament of the invention for use in the prevention or treatment of an unwanted immune reaction against a therapeutic protein.

The invention also relates to a method for preventing or treating an unwanted immune reaction against a therapeutic protein in a subject in need thereof, comprising administering to said patient clinical-grade CD8⁺ CD45RC^{low/-} Treg cells or cell population of the invention, or to the composition, pharmaceutical composition, or medicament of the invention.

As used herein, the term “unwanted immune response against a therapeutic protein” refers to any unwanted immune reaction directed to proteins expressed in the course of gene therapy, and/or therapeutic proteins, such as factor VIII (hemophilia A) and other coagulation factors, enzyme replacement therapies, monoclonal antibodies (e.g. natalizumab, rituximab, infliximab), polyclonal antibodies, enzymes or cytokines (e.g. IFNβ).

In another embodiment, the invention relates to clinical-grade CD8⁺ CD45RC^{low/-} Treg cells or cell population of the invention, or to the composition, pharmaceutical composition, or medicament of the invention for use in the prevention or treatment of an allergy.

The invention also relates to a method for preventing or treating an allergy in a subject in need thereof, comprising administering to said subject clinical-grade CD8⁺ CD45RC^{low/-} Treg cells or cell population of the invention, or to the composition, pharmaceutical composition, or medicament of the invention.

As used herein, the term “allergy” or “allergies” refers to a disorder (or improper reaction) of the immune system. Allergic reactions occur to normally harmless environmental substances known as allergens; these reactions are acquired, predictable, and rapid. Strictly, allergy is one of four forms of hypersensitivity and is called type I (or immediate) hypersensitivity. It is characterized by excessive activation of certain white blood cells called mast cells and basophils by a type of antibody known as IgE, resulting in an extreme inflammatory response. Common allergic reactions include eczema, hives, hay fever, asthma, food allergies, and reactions to the venom of stinging insects such as wasps and bees.

In some embodiments, the subject was previously transplanted, or will be transplanted. In some embodiments, the subject is affected, preferably is diagnosed, with an autoimmune disease. In some embodiments, the subject is affected, preferably is diagnosed, with a chronic inflammatory disease. In some embodiments, the subject previously received a therapeutic protein, or will receive a therapeutic protein. In some embodiments, the subject is affected, preferably is diagnosed with allergy.

In some embodiments, the subject is treated with an immunosuppressive drug, such as, for example, rapamycin, cyclosporine A, mycophenolate mofetil, methylprednisolone, tacrolimus or combinations thereof.

In other embodiments, the subject is not treated with an immunosuppressive drug.

### BRIEF DESCRIPTION OF THE DRAWINGS

FIG. 1 shows the percentage (A) and the mean fluorescence intensity (MFI) (B) of CD45RC^{neg} in CD8⁺ T cells stained with a range of anti-CD45RC mAbs. Triangle: GMP-compatible clone generated and labelled with FITC following ISO's norm 13485. Circle: commercially available non-GMP anti-CD45RC mAb MT2 clone. (C) Representative staining of PBMCs with GMP-compatible anti-CD45RC-FITC mAbs (left) or non-GMP clone (right) and anti-CD8 mAbs. (D) Cell count obtained from 150 mL of blood from healthy volunteers after Ficoll gradient or in total blood (without Ficoll separation). (E) Count of CD8⁺ cells isolated by magnetic beads using Ficoll gradient and LS column or the CliniMACS® system on total blood. (F) Purity of CD8⁺ cells obtained after magnetic isolation using Ficoll gradient and LS column or the CliniMACS® system on total blood. (G) CD8⁺ CD4⁻ CD56⁻ CD45RC^{low/-} cell count obtained by MACSQuant® Tyto® cell sorter from CD8⁺ cell fraction enriched by Ficoll and LS column or by CliniMACS® system. (H) Frequency of CD8⁺ CD56⁻ CD4⁻ CD45RC^{low/-} Treg cells in cell suspension before and after sorting using the MACSQuant® Tyto® sorter from CliniMACS® selected cells (n=6) or from Ficoll and LS selected cells (n=8).

FIG. 2 shows different stimulations of CD8⁺ Treg cells. (A) CD8⁺ Treg cells were stimulated with a range of coated anti-CD3 and soluble anti-CD28 mAbs (down triangle: homemade from ECACC hybridoma; circle: GMP from Miltenyi, dose of anti-CD3/anti-CD28 mAbs in μg/mL are indicated) or with MACS GMP ExpAct Treg kit from Miltenyi (triangles up) and cultured for 14 days in RPMI1640 culture medium supplemented with 10% human AB serum and analyzed for expansion yield (y axis) and suppressive activity (x axis). Dotted lines indicate expansion and suppression scores of CD8⁺ Treg cells stimulated with home-made mAbs. (B) Phenotypic analysis of Treg cells expanded for 14 days in different stimulation conditions. n=3-6.

FIG. 3 is a set of graphs showing the identification of clinical compatible culture medium for CD8⁺ Treg cells. CD8⁺ Treg cells from healthy volunteers were cultured for 14 days in different culture conditions and compared for expansion yield and suppressive activity on responder T cells stimulated by allogeneic APCs. (A) CD8⁺ Treg cells were cultured for 14 days in 10 different serum-free culture media not supplemented with serum, with 50 nM rapamycin, and analyzed for expansion capacity (y axis) and suppressive capacity (x axis) as compared to CD8⁺ Treg cells cultured in RPMI1640 medium supplemented with 10% human AB serum (dotted lines) and not supplemented (open diamond). (B) CD8⁺ Treg cells were cultured for 14 days in 5 different serum-free culture media supplemented with 10% human AB serum (1: from Sigma, 2: from BioIVT), 5% human albumin (3: Vialebex) or 5% CTS™ serum replacement (4: CTS SR) and with 50 nM rapamycin and analyzed for expansion capacity and suppressive capacity as compared to CD8⁺ Treg cells cultured in RPMI1640 medium supplemented with 10% human AB serum (dotted lines). For (A) and (B), symbols represent the mean of 5 to 8 experiments for each condition. (C) Phenotypic analysis of Treg cells expanded for 14 days in different culture media supplemented with human AB serum and 50 nM rapamycin. n=3-6. (D-G) CD8⁺ Treg cells were cultured in RPMI1640 culture medium supplemented with 10% human AB serum and with or without a range of rapamycin doses from 1 to 100 nM and with a high (1000 U/mL) or a low (25 U/mL) dose of IL-2 and analyzed for expansion yield (D-F: left y axis), suppressive activity (D-E: right y axis; F: x axis) and FOXP3 expression (G). D. n=3-8. Mann Whitney test vs without rapamycin, *p<0.05. Squares=proliferation, circles=expansion. E. n=3-8. Mann Whitney test, *p<0.05. black bars=expansion, white bars=proliferation. F. Mean of 3 to 6 experiments is represented for each condition. Numbers indicate dose of rapamycin; circles: 25 U/ml IL-2; triangles: 1000 U/mL IL-2; dotted lines indicate expansion and suppression scores of CD8⁺ Treg cells cultured in medium supplemented with 1000 U/ml IL-2 and no rapamycin. G. n=8. Two-way ANOVA RM. (H) CD8⁺ Treg cells were cultured in RPMI1640 culture medium supplemented with 10% human AB serum and with or without TGF-β and/or IL-10 and analyzed for expansion yield (left y axis, black bars) and suppressive activity (right y axis, white bars). n=3, ns.

FIG. 4 is a graph showing the expansion yield of CD8⁺ Treg cells. Cell number of CD8⁺ Treg cells isolated from healthy volunteers obtained after 7- and 14-days culture. Dotted line shows the minimum of cell to per cm² in a flask to launch an expansion.

FIG. 5 is a set of graphs showing the feasibility of CD8⁺ Treg cells therapy in kidney failure patients. (A) PBMC count obtained by gradient centrifugation using the lymphocytes separation medium (Eurobio) from 50 mL of blood harvested from healthy volunteers (n=11) or patients with kidney failure (n=5) and dialysis (n=6) in EDTA tubes. Mann Whitney test, **p<0.01. (B) CD8⁺ cell count isolated using CliniMACS® CD8 GMP microbeads and LS column from PBMCs of healthy volunteers (n=7) or patients with kidney failure (n=5) and dialysis (n=6). Mann Whitney test. (C) Purity of CD8⁺ cells obtained using CliniMACS® CD8 GMP microbeads system. (D) CD8+ Treg cells (CD4⁻ CD56⁻ CD45RC^{low/-} cells) count isolated from 50 mL blood from healthy volunteers (n=7) or patients with kidney failure (n=5) and dialysis (n=6). Mann Whitney test, **p<0.01. (E) Frequency of CD8⁺ CD4⁻ CD56® CD45RC^{low/-} cells in PBMCs before sorting and in positive (sorted) and negative (remaining) subsets after sorting. (F) Cell count obtained after 7-, 14- and 21-day culture for patients and healthy volunteers.

FIG. 6 is a set of graphs showing the identification of clinical compatible culture media for CD8⁺ Treg cells. CD8⁺ Treg cells from healthy volunteers were cultured for 14 days in different culture conditions and compared for expansion yield and suppressive activity on responder T cells stimulated by allogeneic APCs. (A) CD8⁺ Treg cells were cultured for 14 days in 5 different serum-free culture media supplemented or not with serum (10% human AB serum from Sigma (1: AB serum) or 5% CTS™ serum replacement (4: CTS SR)), with or without 50 nM rapamycin (R), and analyzed for expansion capacity (y axis) and suppressive capacity (x axis) as compared to CD8⁺ Treg cells cultured in RPMI1640 medium which was given the x and y values 1. (A) LymphoONE™, (B) X-VIVO™ 15, (C) ImmunoCult™-XF, (D) PRIME-XV® and (E) CTS™ OpTmizer™. Symbols represent the mean of 5 to 8 experiments for each condition.

FIG. 7 is an MA plot showing the log 2 (fold-change) in function of the mean expression of each gene. Genes differentially expressed are represented by a black dot. Top genes based on p-value are named: 18 genes are underexpressed (upper part of the graph) and 30 genes are overexpressed (lower part of the graph) in cells cultured in X-VIVO™ 15 compared to RPMI 1640 medium.

FIG. 8 is a set of two graphs showing expansion fold of CD8⁺ Treg cultured either in (A) LymphoONE™ (L-), (B) X-VIVO™ 15 (X-), or in medium without supplementation (0), or supplemented with human AB serum (1) or CTS™ serum replacement (4), with or without rapamycin (R). n=4-8. Mann Whitney test: *p<0.05, **p<0.01.

The present invention is further illustrated by the following examples.

### EXAMPLES

### Example 1: GMP Cell Culture Conditions

### Material and Methods

### Sample

Blood was collected from healthy donors at the Etablissement Français du Sang (Nantes, France). Blood harvested from healthy volunteers in Ethylenediaminetetraacetic acid (EDTA) tubes was diluted 2-fold with PBS before peripheral blood mononuclear cells (PBMC) were isolated by Ficoll-Paque density-gradient centrifugation (Eurobio, Courtaboeuf, France) at 2000 rpm for 20 min at room temperature without braking. Collected PBMC were washed in 50 mL PBS at 1800 rpm for 10 min and remaining red cells and platelets are eliminated after incubation 5 min in a hypotonic solution and centrifugation at 1000 rpm for 10 min.

### CD8+ Pre-Enrichment

CD8⁺ pre-enrichment was performed in two manners. PBMC were stained with CliniMACS® CD8 GMP microbeads (Miltenyi Biotec) and then isolated using LS column (Miltenyi Biotec) combined to MidiMACS Separator and MACS MultiStand (Miltenyi Biotec). CD8⁺ pre-enrichment can also be performed directly on the blood by staining the blood with anti-CD8 GMP microbeads and by isolating CD8⁺ cells using CliniMACS® Plus System (Miltenyi Biotec). After CD8⁺ cells isolation, purity was assessed by flow cytometry.

### Flow Cytometry

After the pre-enrichment of CD8⁺ cells with CliniMACS® Plus System or using Ficoll and LS column, the cells were stained with clinical grade anti-CD8 (Miltenyi Biotec), anti-CD4 (Miltenyi Biotec), anti-CD56 (Miltenyi Biotec) and anti-CD45RC (AbolerIS Pharma) antibodies. CD8⁺ CD4⁻ CD56⁻ CD45RC^{low/-} cells were then isolated using the MACSQuant® Tyto® cell sorter (Miltenyi Biotec). The percentage of CD8⁺ CD4⁻ CD56⁻ CD45RC^{low/-} cells was assessed before sorting and after sorting, in the positive and negative fractions (i.e., cells that were through away with the MACSQuant® Tyto® cell sorter).

CD8⁺ CD56⁻ CD4⁻ CD45RC^{low/-} Treg cells were immunophenotyped using anti-GITR (Miltenyi Biotec), anti-HLA-DR (BD Biosciences), anti-CD28 (BD Biosciences), anti-CD27 (BD Biosciences), anti-CCR7 (Miltenyi Biotec), anti-PD1 (eBiociences), anti-CD154 (BD Biosciences), anti-CD127 (BD Biosciences), anti-CD45RA (BD Biosciences), anti-TGF-β (BD Biosciences), anti-IL-34 (R&D Biotechne), anti-IFN-γ (BD Biosciences), anti-Foxp3 (BD Biosciences) and anti-IL-10 (BD Biosciences) antibodies.

For Foxp3+, TGFbeta, IL-34, IFN-γ and IL-10 staining, CD8⁺ CD56⁻ CD4⁻ CD45RC^{low/-} Treg cells were permeabilized using Fixation/Permeabilization kit (eBioscience) prior to antibody staining.

### Expansion Cell Culture

Treg cells were seeded in research grade medium RPMI1640 or in clinical grade serum-free medium Biotarget (Cliniscience), TexMACS (Miltenyi Biotech), X-VIVO™ 15 (Lonza), CTS™ AIM V (ThermoFisher Scientific), CTS™ OpTmizer™ (ThermoFisher Scientific), LymphoONE™ (Takara), PRIME-XV (Irvine Scientific), Stem cell growth medium (SCGM) (Cellgenix) and ImmunoCult™-XF (Stem cell technologies). Treg cell culture media were supplemented with clinical grade cytokines IL-2 at 25 U/mL or 1000 U/mL (Novartis), IL-15 at 10 ng/mL (Miltenyi Biotec) and rapamycin at 1-100 nM (Pfizer). Treg cell culture media were supplemented or not with 10% human AB serum (Sigma Aldrich or BioIVT/Seralab), 5% human albumin Vialebex (LFB Biomedicaments), 5% CTS™ serum replacement (ThermoFisher Scientific). IL-2 and IL-15 cytokines were freshly added every two days during a 14-day expansion culture. In some experiments, clinical grade TGF-β and/or IL-10 were also added to the culture medium (Mitenyi Biotec) at a concentration of 5 ng/mL.

### Cell Stimulation

Treg cells were stimulated with research or clinical grade coated anti-CD3 mAb at 1, 5 or 10 μg/ml (Miltenyi Biotec), soluble research or clinical grade anti-CD28 mAb at 1, 5 or 10 μg/ml (Miltenyi Biotec). At day 7, expanded cells were stimulated again.

### Cell Density

CD56⁻ CD8⁺ CD4⁻ CD45RC^{low/-} Treg cells were seeded in a 25 cm² flask at different concentration of cells/cm².

### Proliferation

Cells were counted on days 0, 7 and 14 on Malassez and expansion fold was calculated by dividing cell count obtained at day 7 or 14 by cell count seeded at day 0. Cells cultured in RPMI1640 culture medium as a reference was assigned the expansion score value of 1.

### Suppressive Capacities

CFSE-labeled CD4+CD25− effector T cells were cultured with allogeneic APCs pooled from 3 donors in absence or presence of Tregs syngeneic with Teffs at ratio Tregs:Teff:APC 1:1:1. Frequency of Teff proliferation was calculated by dividing the frequency of CFSE^{low} Teffs in presence of Tregs by the frequency of CFSE^{low} Teffs in absence of Tregs. Suppression efficiency was calculated as 100%−Frequency of Teff proliferation, and assigned a value of 1 for Tregs generated in a reference culture medium to obtain a suppression score for each culture condition.

### Results

### CD8⁺ Treg Cell Isolation

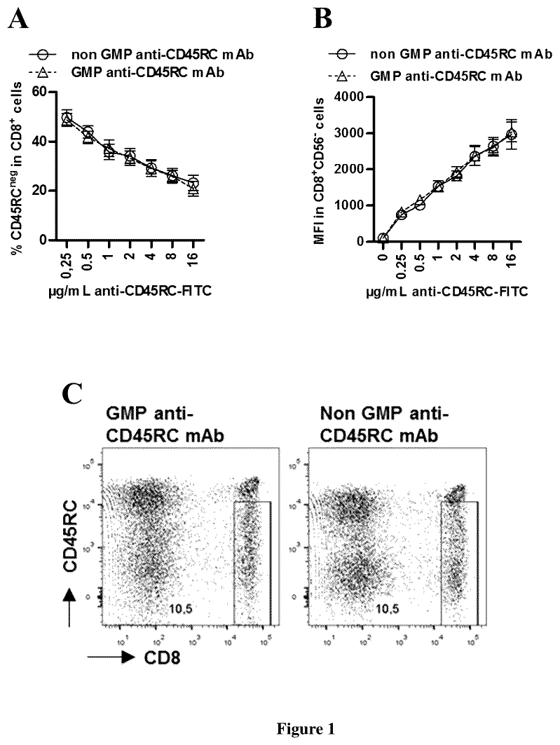

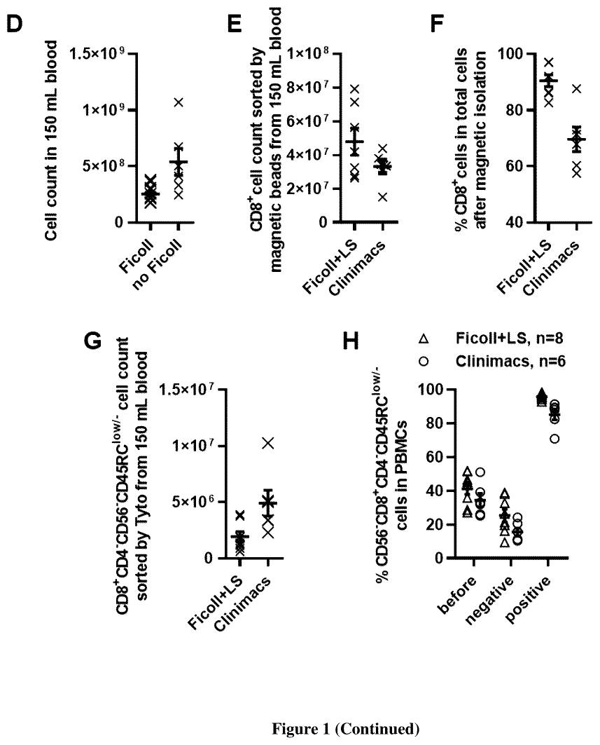

FOXP3⁺ cells are contained both in CD45RC^{low} and CD45RC^{neg} subsets, and there is no functional difference between those subsets. The inventors thus chose to preserve both CD45RC^{low} and CD45RC^{high} subsets and exclude only CD45RC^{high} cells. Depletion of CD45RC^{high} cells can be performed using clinical grade magnetic beads or GMP flow cytometry cell sorter. Both strategies require the production of an anti-CD45RC mAb following ISO's norm 13485. By using magnetic depletion, incubation of PBMCs with increasing doses of anti-CD45RC mAbs allows depletion of CD45RC^{high} but presents limitations regarding the possible elimination of at least a part of CD45RC^{low} cells, the degree of elimination of CD45RC^{high} and also the inherent variability of CD45RC expression among individuals. Thus, a clinical grade compatible flow cytometry cell sorter was used, namely the MACSQuant® Tyto® sorter from Miltenyi, that allows cell sorting under low pressure in a totally closed system, and a GMP-compatible FITC-labelled anti-CD45RC mAbs was generated. This mAbs was validated by flow cytometry staining of CD8⁺ T cells as compared to a commercially available non-GMP anti-CD45RC mAbs clone (FIG. 1. A-C). In the research process, CD8⁺ Tregs used to be sorted on CD56⁻ CD8⁺ CD4⁻ CD45RC^{low/-} expression by FACS Aria IIu (Becton Dickinson) directly from PBMC isolated by Ficoll gradient. In the GMP process, isolation of CD56⁻ CD8⁺ CD4⁻ CD45RC^{low/-} cells by MACSQuant® Tyto® cell sorter required CD8+ cells pre-enrichment using CliniMACS® CD8⁺ beads system but avoided a time-consuming Ficoll gradient step and cell loss (FIG. 1D-E). Without Ficoll gradient step, a higher number of cells were counted in total blood (FIG. 1D) and similar CD8⁺ cell counts and purity were obtained by CliniMACS® system or Ficoll combined to LS column (FIG. 1E-F). Finally, a higher number of CD8⁺ CD4⁻ CD56⁻ CD45RC^{low/-} cells were obtained by MACSQuant® Tyto® cell sorter after CliniMACS® enrichment from total blood compared to Ficoll and LS gradient enriched cells (FIG. 1G), with similar purity (about 90%) and efficiency of Tregs sorting (about 70%) (FIG. 1H).

### Stimulation of CD8+ Treg Cells

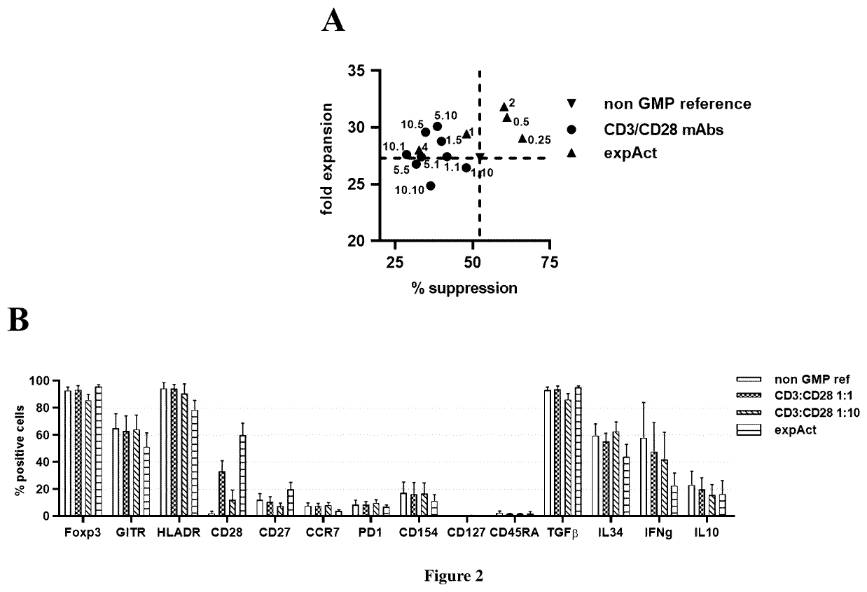

We compared stimuli for CD8⁺ Treg cell culture such as clinical grade purified anti-CD3/CD28 mAbs (from 1 to 10 μg/mL), anti-CD3/CD28 (ExpAct, from 0.25 to 4 doses according to the provider) mAb-coated beads on expansion score and preservation of suppressive function as compared with research grade purified coated anti-CD3 and soluble anti-CD28 mAbs (non GMP reference 1 μg/mL each) (FIG. 2A). Altogether, CD8⁺ Treg cell culture was compatible with both stimuli, but surprisingly lower doses of antibodies and thus lower stimulation than that recommended by the provider based on CD4⁺ Treg cells culture was preferred for CD8⁺ Treg cells culture. CD8⁺ Treg cells stimulation with purified GMP antibodies resulted in phenotype similar to the reference one (FIG. 2B). Thus, the inventors selected purified clinical grade mAbs stimulation to mimic usual research grade stimulation.

### Culture Medium

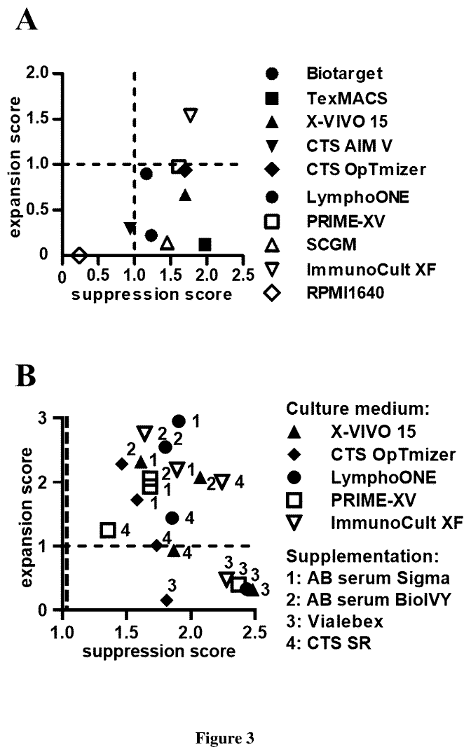

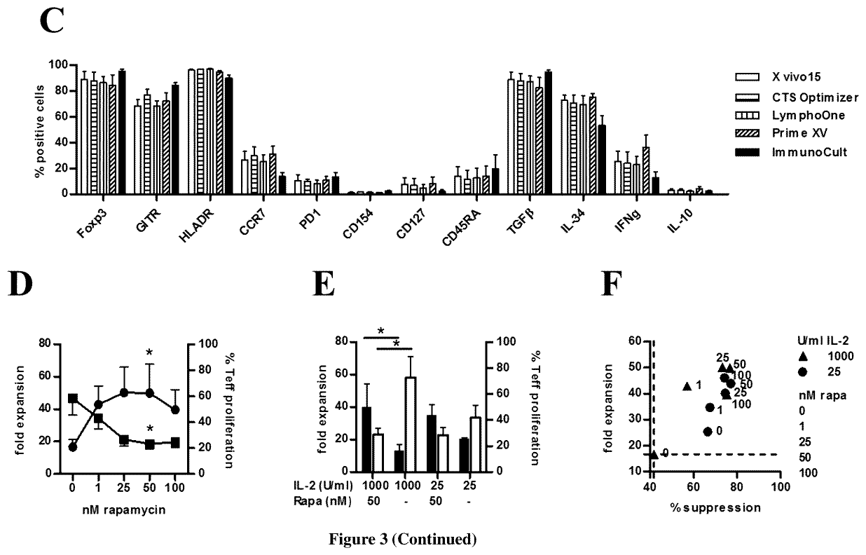

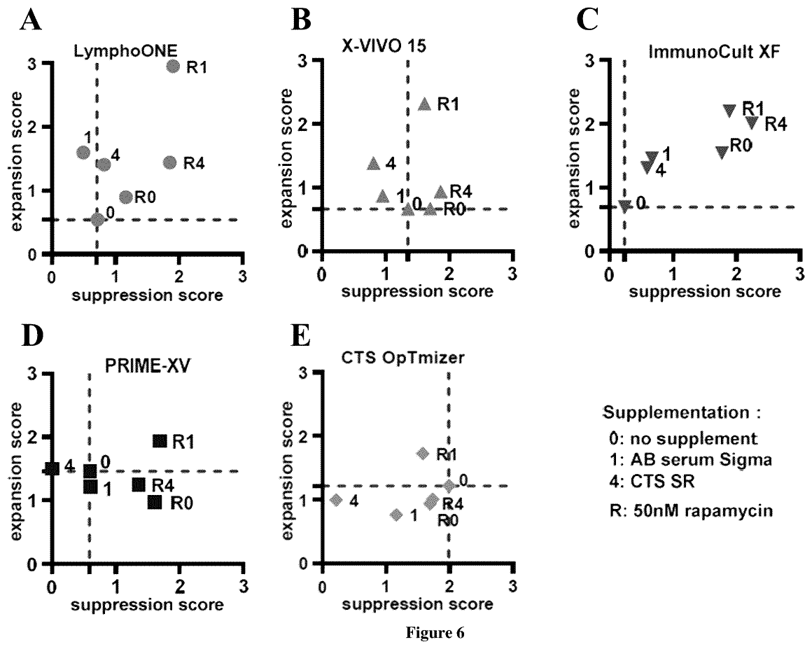

Clinical compatible media and supplements were tested for CD8⁺ CD45RC^{low/-} Treg GMP culture. To be closer to clinical grade conditions, the inventors first assessed expansion and suppression score of CD8⁺ CD45RC^{low/-} Tregs from healthy volunteers in the research grade medium RPMI1640 without serum supplementation. RPMI1640 used in presence of rapamycin, IL-2 and IL-15 but without serum supplementation (FIG. 3A—diamond condition) resulted in a high decrease of expansion and suppression capacity of the cells compared to research grade reference medium in the presence of serum (FIG. 3A—dotted lines). The inventors thus assessed the expansion and suppressive abilities of CD8⁺ Treg cells in culture conditions using ten clinical grade serum-free culture media. ImmunoCult™-XF, PRIME-XV, CTS™ OpTmizer™, X-VIVO™ 15 and LymphoONE™ are culture grade media which confer to the CD8⁺ Treg cells at least the same or a much higher expansion and suppressive abilities than the RPMI1640 medium (FIG. 3A and FIG. 6A-E).

Addition of 50 nM of rapamycin in all culture media highly increased suppressive capacities of cells up to 2.7-fold and preserved, even increased, proliferation yield (data not shown). Addition of human AB serum greatly improved both expansion and suppression, while human albumin preserved suppressive function but did not allow high-scale production (FIG. 3B and FIG. 6A-E). Finally, the CTS™ immune cell serum replacement was beneficial for expansion and suppressive function.

Culture of CD8⁺ Tregs in LymphoONE™ or X-VIVO™ 15 medium supplemented with both rapamycin and human AR serum (R1) showed a significant increased expansion fold compared to the medium supplemented with only rapamycin (R0) or only human AB serum (1), highlighting a synergistic effect when the cells are cultured in the presence of 1) LymphoONE™ or X-VIVO™ 15 medium, 2) rapamycin and 3) human AB serum (FIG. 6A-B). This shows a synergy of rapamycin and serum with both culture media to reach a high expansion yield, which is critical for a clinical use.

Similar increased expansion fold was also observed when CD8⁺ Tregs were cultured in ImmunoCult™-XF, PRIME-XV or CTS™ OpTmizer™ medium supplemented with both rapamycin and human AR serum (R1) compared to the medium supplemented with only rapamycin (R0) or only human AR serum (1) (FIG. 6C-E).

Moreover, culture of CD8⁺ Tregs in LymphoONE™ medium supplemented with both rapamycin and human AB serum (R1) showed an increased suppression fold compared to the medium supplemented with only rapamycin (R0) or only human AB serum (1), highlighting a synergistic effect when the cells are cultured in the presence of 1) LymphoONE™ medium, 2) rapamycin and 3) human AB serum (FIG. 6A).

Similar increased suppression fold was also observed when CD8⁺ Tregs were cultured in ImmunoCult™-XF medium supplemented with both rapamycin and CTS™ immune cell SR (R4) compared to the medium supplemented with only rapamycin (R0) or only CTS™ immune cell SR (4) (FIG. 6C).

Altogether, LymphoONE™, JmmunoCult™-XF, PRIME-XV®, and X-VIVO™ 15 culture media, particularly when supplemented with 50 nM rapamycin and human AB serum or CTS™ immune cell SR, were preferred for CD8⁺ Treg cells culture (FIG. 3B and FIG. 6A-E). In addition, CD8⁺ Treg cells phenotype was unchanged within these five culture medium conditions (FIG. 3C).

The inventors further investigated the best suitable dose of rapamycin for CD8⁺ Treg cell expansion and preserved function. They observed a dose dependent correlation of CD8⁺ Treg cell expansion and suppression with rapamycin dose, reaching a plateau for 50 nM rapamycin (FIG. 3D). Furthermore, lower dose of IL-2 was tested, i.e., 25 U/mL instead of 1000 U/mL. Higher suppression was observed in presence of 25 U/mL vs 1000 U/mL IL-2 when combined with a low dose of rapamycin (i.e., 0-1 nM) suggesting a preferred dose of 25 U/mL IL-2 by Tregs. When combined with a high dose of rapamycin (>25 nM), the addition of 25 or 1000 U/mL IL-2 resulted in similar expansion and suppression which were globally higher than with <25 nM rapamycine combined with 25 or 1000 U/mL IL-2 (FIG. 3E-F). In addition, and also unexpectedly, FOXP3 expression was slightly higher in CD8⁺ Treg cells cultured with 25 U/mL IL-2 than with 1000 U/mL IL-2 (FIG. 3G).

As TGFβ was reported for CD4⁺ CD127⁻ CD25⁺ Tregs expansion to stabilize FOXP3 expression, the benefit of adding 5 ng/mL TGF-β for the culture of CD8⁺ Treg cells was assessed but the inventors did not observe any significative difference (FIG. 3H). Similarly, supplementation of culture medium with 5 ng/mL IL-10 did not affect CD8+ Treg cells proliferation and function (FIG. 3H).

### Cell Density

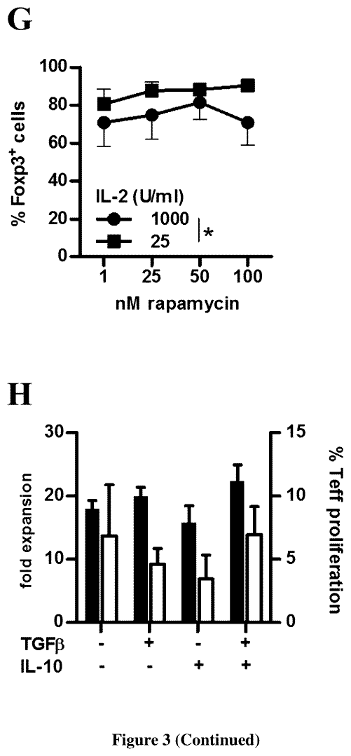

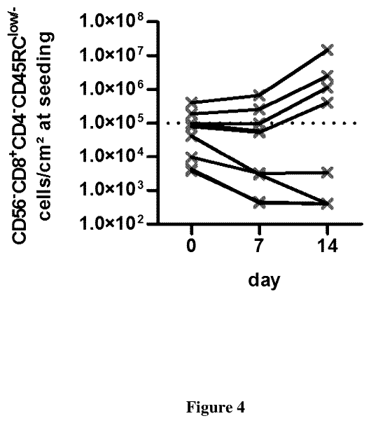

To determine the density suitable for seeding CD8⁺ Treg cells in the GMP culture conditions, different cell numbers were seeded in 25 cm² flasks and analyzed their proliferation. Seeding less than 1×10⁵ cells/cm² resulted in high cell death of the cells or low proliferation (FIG. 4).

### Example 2: CD8⁺ Treg Cells Isolation in Kidney Failure Patients

### Material and Methods

### Samples

PBMC were obtained by gradient centrifugation using the lymphocyte separation medium (Eurobio) from 50 mL of blood harvested from healthy volunteers (n=11) or patients with kidney failure (n=5) and dialysis (n=6) in Ethylenediaminetetraacetic acid (EDTA) tubes.

### CD8⁺ Pre-Enrichment

CD8⁺ pre-enrichment was performed directly on the blood by staining the blood with anti-CD8 GMP microbeads and by isolating CD8⁺ cells using CliniMACS® Plus (Miltenyi Biotec).

### Flow Cytometry

After pre-enrichment of CD8⁺ cells with CliniMACS® Prodigy, the cells were stained with anti-CD8 (Miltenyi Biotec), anti-CD4 (Miltenyi Biotec), anti-CD56 (Miltenyi Biotec) and anti-CD45RC (AbolerIS Pharma) antibodies. CD8⁺ CD4⁻ CD56⁻ CD45RC^{low/-} cells were then isolated using the MACSQuant® Tyto® cell sorter (Miltenyi Biotec). The percentage of CD8⁺ CD4⁻ CD56⁻ CD45RC^{low/-} cells was assessed before sorting and in positive (sorted) and negative (remaining) subsets after sorting by flow cytometry.

### Cell Culture

CD8⁺ CD4⁻ CD56⁻ CD45RC^{low/-} cells were cultured in X-VIVO™ 15 culture medium (Lonza) supplemented with 5% CTS™ immune cell serum replacement (ThermoFisher scientific), 50 nM rapamycine (Pfizer), 25 U/mL IL-2 (Novartis) and 10 ng/mL IL-15 (Miltenyi Biotec). CD8⁺ CD4⁻ CD56⁻ CD45RC^{low/-} cell count was assessed after 7, 14 and 21 days of culture for patients and healthy volunteers on Malassez.

### Results

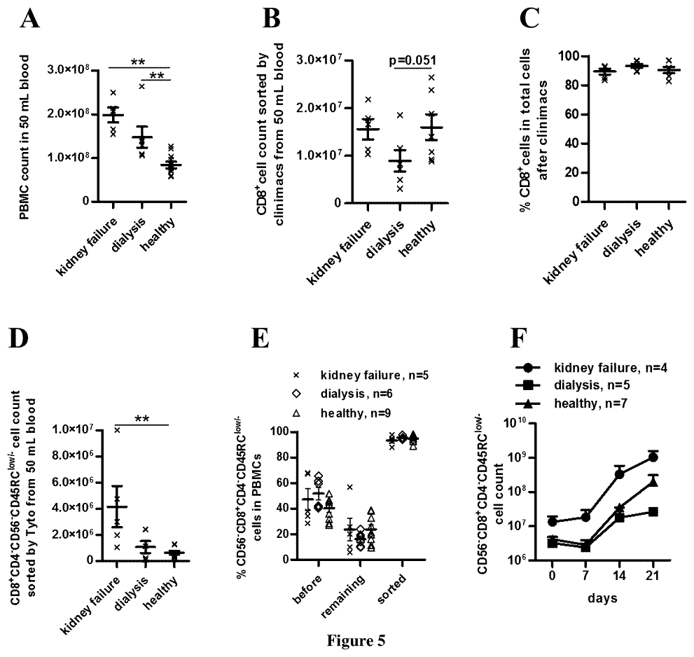

After defining ideal clinical grade culture parameters for CD8⁺ Treg cell production from blood of healthy volunteers, the feasibility of generating CD8⁺ Tregs from patients with kidney failure was assessed. Among them, 6 were under dialysis and 5 were not, as compared with healthy volunteers. Surprisingly, significantly more PBMCs were isolated from 50 mL blood of kidney failure patients than from healthy volunteers (FIG. 5A), but similar CD8⁺ cell count (FIG. 5B). Positive selection of CD8⁺ T cells using magnetic beads aimed to reduce the time of subsequent flow cytometry cell sorting and improved purity of CD8⁺ cells up to 97% (FIG. 5C), enriched in CD8⁺ Treg cells from 3% up to 50%, and improved final yield of sorted CD8⁺ Treg cells up to 3-fold. Starting from 50 mL blood, a mean of 1.3×10⁶, 1.07×10⁶ and 4.16×10⁶ CD8⁺ CD4⁻ CD56⁻ CD45RC^{low/-} T cells were sorted from healthy volunteers, dialysis patients and kidney failure patients respectively (FIG. 5D). Purity of sorted CD8⁺ Tregs was greater than 98% for both healthy volunteers and patients (FIG. 5E). Finally, CD8⁺ Tregs from kidney failure patients expanded as well as cells from healthy volunteers (FIG. 5F).

### Example 3: Comparison of CD8⁺ Tregs Cultured in X-VIVO™ 15 vs. RPMI 1640 Medium by RNA Sequencing

### Material and Methods

### Isolation of Peripheral Blood Mononuclear Cells (PBMCs)

Blood was harvested from healthy volunteers, PBMCs were isolated by Ficoll-Paque density-gradient centrifugation (Eurobio, Courtaboeuf, France) at 2000 rpm for 20 min at room temperature without braking. Collected PBMCs were washed in 50 mL PBS at 1800 rpm for 10 min and remaining red cells and platelets were eliminated after 5 min incubation in a hypotonic solution and centrifugation at 1000 rpm for 10 min.

### Isolation and Initiation of Culture of CD8⁺ Tregs

PBMCs were resuspended at 2×10⁸ cells/ml in PBS-FCS-EDTA and incubated with anti-CD3-PeCy7 (BD Biosciences), anti-CD4-PerCPCy5.5 (BD Biosciences) and anti-CD45RC-FITC (MT2 clone, IqProduct) mAbs for 30 min at 4° C. Cells were washed with PBS-FCS-EDTA, filtered on 60 μm tissue, labeled with Dapi and FACS Aria II (BD Biosciences)-sorted on lymphocyte morphology, exclusion of doublet cells and DAPI-CD3⁺ CD4⁻ CD45RC^{low/-} expression.

After sorting, cells were seeded at 10⁵ Tregs/cm²/300 μL in plate previously coated with anti-CD3 mAbs (OKT3 clone, 1 μg/ml in PBS, 100 μL/cm², 1 h at 37° C., then washed with PBS 3 times) in RPMI 1640 culture medium supplemented with 10% human serum, Penicillin (100 U/ml), Streptomycin (0.1 mg/ml), Sodium pyruvate (1 mM), Glutamine (2 mM), Hepes Buffer (1 mM), non-essential amino acids (lx), IL-2 (1000 U/ml, Novartis), IL-15 (10 ng/mL, Miltenyi Biotec) and anti-CD28 mAbs (clone CD28.2, soluble, 1 μg/ml). Cells were incubated at 37° C. 5% CO₂.

### Isolation and Initiation of Culture of CD8⁺ Tregs Using the Method of the Invention

PBMC were stained with CliniMACS® CD8 GMP microbeads (Miltenyi Biotec), CD8⁺ cells were isolated using LS column (Miltenyi Biotec) combined to MidiMACS Separator and MACS MultiStand (Miltenyi Biotec), then stained with anti-CD8-APC (Miltenyi Biotec), anti-CD4-VioBlue (Miltenyi Biotec), anti-CD56-PE (Miltenyi Biotec) and anti-CD45RC-FITC (ABIS-45RC clone, AbolerIS Pharma) mAbs. CD8⁺ CD4⁻ CD56⁻ CD45RC^{low/-} cells were isolated using the FACS Aria II cell sorter (BD Biosciences).

After sorting, cells were washed in medium, seeded at 10⁵ Tregs/cm²/300 μL in plate previously coated with anti-CD3 mAb (OKT3 clone, 1 μg/ml in PBS, 100 μL/cm², 1 h at 37° C., then washed with PBS 3 times), in clinical grade serum-free medium X-VIVO™ 15 (Lonza) supplemented with 10% human AB serum (Sigma Aldrich), IL-2 (25 U/mL, Novartis), IL-15 (10 ng/mL, Miltenyi Biotec), rapamycin at 50 nM (Pfizer) and anti-CD28 mAb (clone CD28.2, soluble, 1 ug/ml). Cells were incubated at 37° C. 5% CO₂.

### Culture Maintenance

At day 7, cells were harvested, counted, washed, and seeded at 5×10⁴ Tregs/cm²/300p L in culture plates previously coated with anti-CD3 mAb (OKT3 clone, 1 μg/ml in PBS, 100 μL/cm², 1 h at 37° C., then washed with PBS 3 times), in the same culture medium as used at day 0. Cells were incubated at 37° C. 5% CO₂.

Cytokines were freshly added every 2 days and fresh medium was added when required, depending on proliferation rate.

### RNA Sequencing

CD8⁺ Tregs cultured in RPMI 1640 or X-VIVO™ 15 medium were analyzed by bulk and single cell RNA sequencing respectively. Single cell RNAseq data were converted to a pseudo bulk data matrix, and only genes present in both datasets were kept for further differential gene expression analysis. Differentially expressed genes were determined using the contrast function, with secondary testing (FDR) to obtain q value. Genes were considered differentially expressed if the FDR q value <0.05. Principal component analysis 1 and 2 explained 13% and 65% of variance between the 2 groups. MA plot was drawn by plotting log 2 (fold-change) in function of the mean expression of each gene.

### Tables of Results

TABLE 1
List of genes differentially expressed in CD8⁺Tregs
cultured in X-VIVO ™ 15 vs. RPMI 1640

| Underexpressed | Overexpressed |
|---|---|
| genes | genes |
| IL8 | FMNL1 |
| IFNG | FUT7 |
| BIRC3 | WAS |
| CSF2 | PTPRCAP |
| GZMB | MPST |
| CCL3 | CD3E |
| CCL4 | LNPEP |
| CD52 | C19orf43 |
| B2M | GNB2 |
| FTL | MIIP |
| WARS | BAX |
| GZMA | TGFB1 |
| PSME2 | CORO1B |
| ACAA2 | GZMM |
| UQCR11 | GTF2I |
| CD59 | SLFN12L |
| MYL6 | HIST1H2AG |
| ARPC4 | PRRC2A |
| MINOS1 | PGLS |
| ALDOA | TLN1 |
| SNRPD1 | LY6E |
| SNRPE | ARHGEF1 |
| CKS2 | TPT1 |
| ENO1 | GYPC |
| HMGB1 | CCDC107 |
| PTMA | CSNK1G2 |
| TUBA1B | EMC6 |
| HSPD1 | TMEM183A |
| CYCS | PTMS |
| HSPE1 | AP1S1 |
| SELK | MFSD10 |
| SRGN | CCR8 |
| NDUFA12 | ATP5E |
| SUB1 | FKBP8 |
| TXN | BST2 |
| ERH | MZT2B |
| SLIRP | C10orf128 |
| PSMB3 | HIST1H1E |
| PSMB6 | C9orf16 |
| RPS20 | JOSD2 |
| ATP5I | PTGER4 |
| RPL26 | STUB1 |
| CALM2 | HDGFRP2 |
| ATP5J | RALY |
| COX6C | RBBP4 |
| SRP9 | RHOF |
| ATP5O | DPP7 |
| SHFM1 | SREBF2 |
| NDUFA4 | RPL13 |
| SKP1 | GLTSCR2 |
| TMEM258 | JUND |
| RPL31 | ABHD17A |
| GHITM | FASN |
| RBX1 | EMP3 |
| RPL35 | SRSF9 |
| UBB | SF3A2 |
| IER3IP1 | C20orf24 |
| PSMA4 | TSR3 |
| UQCRQ | TPGS1 |
| SNRPD2 | LIMD2 |
| CBX3 | CORO1A |
| LSM3 | H3F3A |
| NDUFV2 | NUDT3 |
| RPL7 | CCDC85B |
| SNRPG | JUN |
| NME1 | MZT2A |
| TOMM6 | PRELID1 |
| UBE2L3 | DRAP1 |
| NDUFA6 | ATP5D |
| RPL36AL | RPL21 |
| NEDD8 | SOCS1 |
| NOP10 | H1FX |
| CHMP4A | PNPLA2 |
| NDUFC2 | PRKCSH |
| CLDND1 | PABPN1 |
| C11orf48 | SNRPD3 |
| NDUFA7 | DDA1 |
| PSMA2 | VTI1B |
| PSMA1 | EIF4A1 |
| TOMM5 | THOC3 |
| ARL6IP1 | PYURF |
| COX6A1 | C7orf55-LUC7L2 |
| ECH1 | MIR142 |
| TMA7 | MTPN |
| CSNK2B | TRAPPC1 |
| NDUFA11 | CD7 |
| PSMA6 | S1PR4 |
| PRR13 | FXYD5 |
| SQRDL | TNFRSF18 |
| RPL23 | RPS26 |
| GALM | HIST2H2AC |
| SARNP | PPAP2A |
| SRSF10 | DGKZ |
| EEF1G | EEF1D |
| NDUFA13 | SSBP4 |
| NDUFB8 | HSF1 |
| H3F3B | TMEM259 |
| LGALS1 | AES |
| CD2 | CFL1 |
| CD3D | GPSM3 |
| TMSB4X | CCNI |
| SERF2 | FADS2 |
| UQCR10 | LSP1 |
| RPS19 | TSC22D4 |
| SDHD | EEF2 |
| TMBIM4 | MAP2K2 |
| LCP1 | PPDPF |
| TMBIM6 | MYO1G |
| RPL15 | RABL6 |
| COX7A2 | EHBP1L1 |
| DBI | GRINA |
| RPS29 | AHNAK |
| POMP | HLA-C |
| PSMB1 | ARHGDIA |
| HMGN2 | VIM |
| ATP5L | ARHGAP4 |
| TCEB2 | SMARCB1 |
| ARHGDIB | FLNA |
| GMFG | GRAMD1A |
| RPS27 | RPS4Y1 |
| RPL34 | FSCN1 |
| RPS14 | JUNB |
| RPS23 | MIF |
| RPS18 | UBE2S |
| RPL3 | CD70 |
| COX17 | LMNA |
| RPS6 | RPSAP58 |
| PARK7 | HIST1H2AL |
| HMGB2 | SYNC |
| SLC25A5 | PPP1R14B |
| | SRM |
| | RPSA |
| | PKN1 |
| | PLEC |
| | EIF5A |
| | NME1-NME2 |
| | TYMS |

TABLE 2
List of genes differentially expressed in CD8⁺
Tregs cultured in X-VIVO ™ 15 medium vs.
RPMI1640 with known function in inflammation or tolerance

| | | Up-regulated or | Known |
|---|---|---|---|
| | | down-regulated | function in relation |
| | | (X-VIVO ™ | with inflammation |
| Gene | Full name | 15 vs RPMI) | or tolerance |
| TGFβ1 | Transforming | up-regulated | protein secreted by CD8⁺ |
| | growth factor | | Tregs that participate to |
| | beta-1 | | the suppressive activity |
| | | | of the cells |
| GITR | Glucocorticoid- | up-regulated | protein expressed by the |
| | Induced TNFR- | | most suppressive CD8⁺ |
| | Related Protein | | CD45RC^{low/-} T cells |
| PKN1 | Serine/threonine- | up-regulated | overexpressed in |
| | protein kinase N1 | | FOXP3⁺ CD8⁺ Tregs |
| | | | (versus FOXP3⁻) |
| | | | in rats |
| LSP1 | Lymphocyte- | up-regulated | role in activation and |
| | specific protein 1 | | chemotaxis of M2 |
| | | | macrophages, |
| | | | neutrophils and |
| | | | CD4⁺ Tregs |
| CCR8 | C-C Motif | up-regulated | expressed by |
| | Chemokine | | tumor-infiltrating |
| | Receptor 8 | | CD4⁺ Tregs |
| GzmA, | Granzymes A | down-regulated | associated with |
| GzmB | and B | | cytolysis |
| CCL3, | C-C motif | down-regulated | role in chemotaxis |
| CCL4, | chemokine | | and inflammation |
| IL8 | Ligand 3,4 and | | |
| | Interleukin 8 | | |
| IFNγ | Interferon | down-regulated | pro-inflammatory or |
| | gamma | | pro-regulatory role |
| | | | depending on the |
| | | | proteins present in |
| | | | the environment |

TABLE 3
List of major differentially-expressed genes

| | | Up-regulated or | |
|---|---|---|---|
| | | down-regulated | |
| | | (X-VIVO ™ | |
| Gene | Name/Role | 15 vs RPMI) | Known function |
| EEF1G | Elongation factor 1-gamma | down-regulated | gene expression |
| PSMA2 | Proteasome subunit alpha type-2 | down-regulated | gene expression |
| NDUFA13 | NADH dehydrogenase [ubiquinone] 1 | down-regulated | mitochondria/ |
| | alpha subcomplex subunit 13 | | metabolism |
| ECH1 | Delta(3,5)-Delta(2,4)-dienoyl-CoA | down-regulated | mitochondria/ |
| | isomerase, mitochondrial | | metabolism |
| NDUFB8 | NADH dehydrogenase [ubiquinone] 1 | down-regulated | mitochondria/ |
| | beta subcomplex subunit 8, mitochondrial | | metabolism |
| COX6A1 | Cytochrome c oxidase subunit 6A1 | down-regulated | mitochondria/ |
| | | | metabolism |
| HSPE1 | 10 kDa heat shock protein, mitochondrial | down-regulated | mitochondria/ |
| | | | metabolism |
| UBB | Polyubiquitin-B | down-regulated | proteasome/Ag |
| | | | presentation |
| CCL4 | C-C motif chemokine 4, Monokine with | down-regulated | inflammation |
| | inflammatory and chemokinetic properties | | |
| RPL7 | 60S ribosomal protein L7 | down-regulated | gene expression |
| ATP5J | ATP synthase-coupling factor 6, mitochondrial | down-regulated | mitochondria/ |
| | | | metabolism |
| ENO1 | Alpha-enolase | down-regulated | mitochondria/ |
| | | | metabolism |
| RPL35 | 60S ribosomal protein L35 | down-regulated | gene expression |
| FTL | Ferritin light chain | down-regulated | iron homeostasis |
| RPS19 | 40S ribosomal protein S19 | down-regulated | gene expression |
| GZMB | Granzyme B, activates caspase-independent | down-regulated | cytolysis/ |
| | pyroptosis when delivered into the target cell. | | suppression |
| | Associated with cytolysis and suppression. | | |
| B2M | Beta-2-microglobulin | down-regulated | proteasome/Ag |
| | | | presentation |
| TMSB4X | Hematopoietic system regulatory peptide | down-regulated | cytosquelette/ |
| | | | migration |
| EIF4A1 | Eukaryotic initiation factor 4A-I, | up-regulated | gene expression |
| | ATP-dependent RNA helicase | | |
| RPSA | 40S ribosomal protein SA | up-regulated | gene expression |
| RPL13 | Ribosomal protein L13 | up-regulated | gene expression |
| VIM | Vimentin, class-III intermediate filament | up-regulated | cytoskeleton/ |
| | found in various non-epithelial cell | | migration |
| TPT1 | Translationally-controlled tumor protein | up-regulated | cytoskeleton/ |
| | involved in calcium binding and | | migration |
| | microtubule stabilization | | |
| PABPN1 | Poly(A) binding protein nuclear 1 | up-regulated | gene expression |
| TGFB1 | Transforming growth factor beta-1 protein | up-regulated | regulation |
| FKBP8 | Peptidyl-prolyl cis-trans isomerase FKBP8 | up-regulated | mitochondria/ |
| | | | metabolism |
| PPDPF | Pancreatic progenitor cell differentiation and | up-regulated | memory cells |
| | proliferation factor, more expressed in | | |
| | memory cells | | |
| TYMS | Thymidylate synthase, contributes to the | up-regulated | mitochondria/ |
| | de novo mitochondrial thymidylate | | metabolism |
| | biosynthesis pathway | | |
| LMNA | Prelamin-A/C; Lamins are components of | up-regulated | cytoskeleton/ |
| | the nuclear lamina | | migration |
| H3F3A | H3.3 histone A | up-regulated | gene expression |
| MIIP | Migration and invasion-inhibitory protein | up-regulated | cytoskeleton/ |
| | | | migration |
| PTMS | Parathymosin, ubiquitous small acidic | up-regulated | proteasome/Ag |
| | nuclear proteins thought to be involved in | | presentation |
| | cell cycle progression, proliferation, and | | |
| | cell differentiation | | |
| FSCN1 | Fascin; Actin-binding protein | up-regulated | cytoskeleton/ |
| | | | migration |
| EMC6 | ER membrane protein complex subunit 6 | up-regulated | |
| TPGS1 | Tubulin polyglutamylase complex subunit 1 | up-regulated | cytoskeleton/ |
| | | | migration |
| SYNC | Syncoilin | up-regulated | cytoskeleton/ |
| | | | migration |
| CORO1B | Coronin-1B; Regulates leading edge | up-regulated | cytoskeleton/ |
| | dynamics and cell motility in fibroblasts | | migration |
| SF3A2 | Splicing factor 3A subunit 2; Involved in | up-regulated | gene expression |
| | pre-mRNA splicing as a component of | | |
| | the splicing | | |
| HIST1H2AG | Histone H2A | up-regulated | gene expression |
| PRRC2A | Protein PRRC2A; May play a role in the | up-regulated | gene expression |
| | regulation of pre-mRNA splicing | | |
| PKN1 | Serine/threonine-protein kinase N1, regulation | up-regulated | cytoskeleton/ |
| | of the intermediate filaments of the actin | | migration |
| | cytoskeleton, cell migration, tumor cell | | |
| | invasion and transcription regulation; more | | |
| | expressed in rat GFP⁺CD8Tregs | | |
| LSP1 | Lymphocyte-specific protein 1; May play | up-regulated | regulation |
| | a role in mediating activation and | | |
| | chemotaxis of M2 macrophages, | | |
| | neutrophils, and regulatory T cells. | | |
| MIF | Macrophage migration inhibitory factor | up-regulated | cytoskeleton/ |
| | | | migration |
| CFL1 | Cofilin-1; Binds to F-actin and exhibits | up-regulated | cytoskeleton/ |
| | pH-sensitive F-actin depolymerizing activity | | migration |
| EEF2 | Elongation factor 2 | up-regulated | gene expression |
| CORO1A | Coronin-1A; May be a crucial component | up-regulated | cytoskeleton/ |
| | of the cytoskeleton of highly motile cells | | migration |
| RPS26 | Ribosomal protein S26 | up-regulated | gene expression |

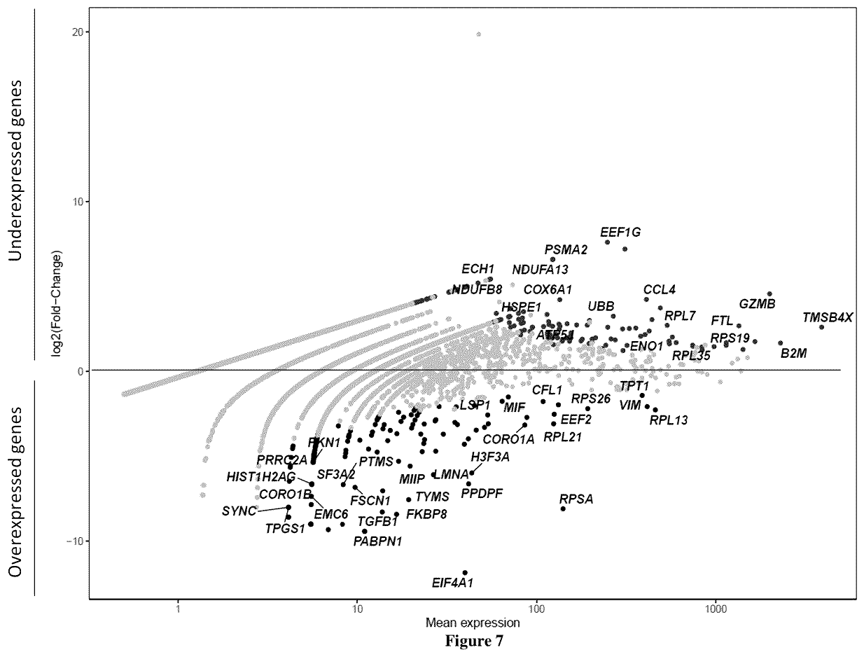

There are 267 differentially expressed (DE) genes between the CD8⁺ Tregs cultured in X-VIVO™ 15 supplemented with rapamyin and human serum vs. RPMI supplemented with human serum (Table 1). Among the 137 genes overexpressed in CD8⁺ Tregs cultured in X-VIVO™ 15 vs. RPMI (FIG. 7), were found TGFβ1 (Transforming growth factor beta-1) which is directly involved in the suppressive function of the CD8⁺ Tregs as disclosed herein, GITR (Glucocorticoid-Induced TNFR-Related Protein) which has been shown to be associated with the function of the CD8⁺ Tregs as disclosed herein, PKN1 (Serine/threonine-protein kinase N1) which has also been shown to be more expressed by CD8⁺ Tregs FOXP3⁺ (GFP⁺) in rats than FOXP3⁻, LSP1 (Lymphocyte-specific protein 1) which has a role in the activation and chemotaxis of M2 macrophages, neutrophils, and CD4⁺ Tregs, CCR8 (C-C Motif Chemokine Receptor 8) which is expressed by tumor-infiltrating CD4⁺ Tregs (Table 2). Among the 130 genes underexpressed in CD8⁺ Tregs cultured in X-VIVO™ 15 vs. RPMI (FIG. 7), were found GzmA and GzmB (granzymes) which are associated with cytolysis, CCL3, CCL4 and IL8 (C-C motif chemokine Ligand 3,4 and Interleukin 8) which have a role in chemotaxis and inflammation, IFNγ (Interferon gamma) which has pro-inflammatory or pro-regulatory role depending on the proteins present in the environment (Table 2). Other genes related to immunity were found, such as CD3E, CD3D, CD2, CD59, and CD70 which have a role in TCR signal transduction and activation of T cells; CD7 which has a role in the interaction of T cells; and HLAC, B2M, and UBB that may be involved in antigen presentation (Table 3). Other differentially expressed genes are linked to the cytoskeleton, the mitochondria/metabolism, or gene expression (Table 3).

### Example 4: Evaluation of the Expansion Fold of CD8⁺ Tregs Obtained by the Method of the Invention

### Material and Methods

### Sample

Blood was collected from healthy donors at the Etablissement Français du Sang (Nantes, France) in Ethylenediaminetetraacetic acid (EDTA) tubes, was diluted 2-fold with PBS before peripheral blood mononuclear cells (PBMC) were isolated by Ficoll-Paque density-gradient centrifugation (Eurobio, Courtaboeuf, France) at 2000 rpm for 20 min at room temperature without braking. Collected PBMC were washed in 50 mL PBS at 1800 rpm for 10 min and remaining red cells and platelets were eliminated after 5 min incubation in a hypotonic solution and centrifugation at 1000 rpm for 10 min.

### Cell Sorting

PBMC concentration was adjusted at 2×10⁸ cells/ml in PBS-FCS-EDTA and cells were incubated with anti-CD3-PeCy7, anti-CD8-APC and anti-CD45RC-FITC for 15 min 4° C. Cells were washed with PBS-FCS-EDTA, filtered on 60 μm tissue, labeled with Dapi and CD8⁺ Tregs were FACS Aria sorted on lymphocyte morphology, exclusion of doublet cells, and DAPI⁻ CD3⁺ CD8⁺ CD45RC^{low/-} expression.

After sorting, CD8⁺ Tregs were washed in PBS, culture plate was coated with anti-CD3 mAb (OKT3 clone, 1 μg/ml in PBS, 100 μL/cm², 1 h at 37° C., then washed with PBS 3 times), and CD8⁺ Tregs were seeded at 3×10⁵/cm²/600 μL in X-VIVO™ 15 (Lonza) or LymphoONE™ (Takara) culture media supplemented with IL-2 (1000 U/mL, Novartis), IL-15 (10 ng/mL, Miltenyi Biotec) and anti-CD28 mAb (clone CD28.2, soluble, 1 ug/ml), with or without human serum 10% (Sigma Aldrich) or CTS™ serum replacement 5% (ThermoFisher Scientific) and/or rapamycin (50 nM, Pfizer). Cells were incubated at 37° C. 5% CO₂.

### Culture Maintenance

At day 7, cells were harvested, washed, and plated at 5×10⁴ Tregs/cm²/300 μL in culture plate previously coated with anti-CD3 mAb (OKT3 clone, 1 μg/ml in PBS, 100 μL/cm², 1 h at 37° C., then washed with PBS 3 times), in the same culture medium as used at day 0. Cells were incubated at 37° C. 5% CO₂.

Cytokines were freshly added every 2 days and fresh medium was added when required, depending on proliferation rate.

### Proliferation

Expansion fold was calculated by dividing cell count obtained at day 14 by cell count seeded at day 0.

### Results

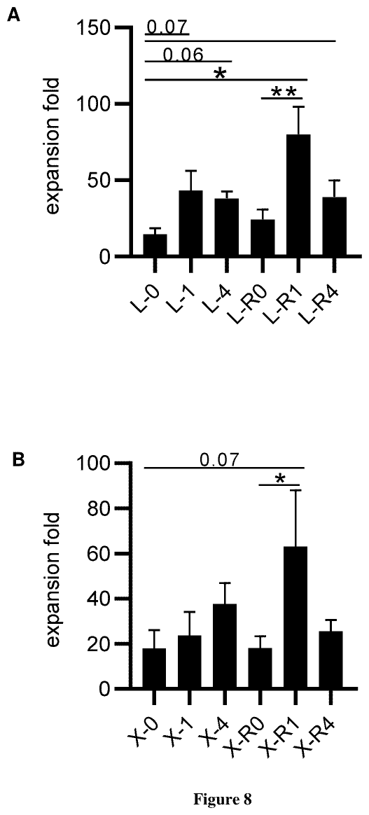

As shown in FIG. 8, culture of CD8⁺ Tregs in LymphoONE™ or X-VIVO™ 15 medium supplemented with rapamycin alone (R0) did not impact their expansion fold. Culture of CD8⁺ Tregs in LymphoONE™ supplemented with human serum alone (L-1) resulted in a trend of increased expansion fold (p value=0.07) (FIG. 8A). Culture of CD8⁺ Tregs in LymphoONE™ medium supplemented with both rapamycin and human serum (L-R1) also showed a significant increased expansion fold compared to the medium with no supplement (L-0) (FIG. 8A). A similar trend was observed for culture in X-VIVO™ 15 medium (X-R1 vs. X-0) (p value=0.07) (FIG. 8B). Culture of CD8⁺ Tregs in LymphoONE™ or X-VIVO™ 15 medium supplemented with both rapamycin and human serum (R1) showed a significant increased expansion fold compared to the medium supplemented with only rapamycin (R0). Compared to the medium with no supplement, culture of CD8⁺ Tregs in LymphoONE™ or X-VIVO™ 15 medium supplemented with both rapamycin and human AB serum (R1) increased expansion fold (p values <0.05 and 0.07 respectively) more efficiently than serum alone (1) (p value=0.0714 and =0.69 respectively) or rapamycine alone (R0) (p value=0.77 and =0.72 respectively). Altogether, these results highlight a synergistic effect when the cells are cultured in the presence of 1) LymphoONE™ or X-VIVO™ 15 medium, 2) rapamycin and 3) human serum (FIG. 8A and FIG. 8B). This shows a synergy of rapamycin and serum with both culture media to reach a high expansion yield, which is critical for a clinical use. Expansion fold of CD8⁺ Tregs was similar when using LymphoONE™ or X-VIVO™ 15 medium, both supplemented with rapamycin and human serum.

### Example 5: Obtention of CD8⁺ Tregs for Cell Therapy

### Material and Methods

### Isolation of PBMCs

Blood is collected from healthy donors at the Etablissement Français du Sang (Nantes, France) in Ethylenediaminetetraacetic acid (EDTA) tubes, diluted 2-fold with PBS, and peripheral blood mononuclear cells (PBMC) are isolated by Ficoll-Paque density-gradient centrifugation (Eurobio, Courtaboeuf, France) at 2000 rpm for 20 min at room temperature without braking. Collected PBMC are washed in 50 mL PBS at 1800 rpm for 10 min and remaining red cells and platelets are eliminated after incubation 5 min in a hypotonic solution and centrifugation at 1000 rpm for 10 min.

### Cell Sorting

Cell concentration is adjusted at 2×10⁸ PBMC/ml in PBS-FCS-EDTA, and cells are incubated with anti-CD3-PeCy7, anti-CD8-APC and anti-CD45RC-FITC mAbs for 15 min 4° C. Cells are washed with PBS-FCS-EDTA, filtered on 60 μm tissue, labeled with Dapi and CD8⁺ Tregs are FACS Aria-sorted on lymphocyte morphology, exclusion of doublet cells, and DAPI⁻ CD8⁺ CD56⁻ CD4⁻ CD45RC^{low/-} expression.

After sorting, cells are washed in PBS, culture plates are coated with anti-CD3 mAb (OKT3 clone, 1 μg/ml in PBS, 100 μL/cm², 1 h at 37° C., then washed with PBS 3 times), and cells are seeded at 3×10⁵/cm²/600 μL in X-VIVO™ 15 (Lonza), LymphoONE™ (Takara), ImmunoCult™-XF, PRIME-XV®, or CTS™ OpTmizer™ culture medium supplemented with IL-2 (25 U/mL, Novartis), IL-15 (10 ng/mL, Miltenyi Biotec), anti-CD28 mAb (clone CD28.2, soluble, 1 ug/ml), human serum 10% (Sigma Aldrich) and rapamycin (50 nM, Pfizer). Cells are incubated at 37° C. 5% CO₂.

### Culture Maintenance

At days 7 and 14, cells are harvested, washed, and plated at 5×10⁴ Tregs/cm²/300p L in culture plate previously coated with anti-CD3 mAb (OKT3 clone, 1 μg/ml in PBS, 100 μL/cm², 1 h at 37° C., then washed with PBS 3 times) in the same culture medium as used at day 0. Cells are incubated at 37° C. 5% CO₂.

Cytokines are freshly added every 2 days and fresh medium is added when required, depending on the proliferation rate.

### Example 6: Evaluation of the Cytotoxic Activity of CD8⁺ Tregs Obtained by the Method of the Invention Against Human Primary Aortic Endothelial Cells

### Material and Methods

Primary human vascular ECs prospectively isolated from kidney transplant donors (Institut de Transplantation Urologie Nephrologie, CHU de Nantes) are stored in liquid nitrogen (DIVAT Sample Biocollection, French Health Ministry project number 02G55). CD8⁺ Tregs obtained by the method described in Example 5 are added on a confluent monolayer of human primary aortic endothelial cells in flat-bottom 96-well plates in a range of ratios from 1:2 to 5:1 T cells: endothelial cells in RPMI 1640 medium supplemented with 10% AB serum, Penicillin (100 U/ml), Streptomycin (0.1 mg/ml), Sodium pyruvate (1 mM), Glutamine (2 mM), Hepes Buffer (1 mM), and non-essential amino acids (lx), plates are centrifuged for 1 minute at 430 g, and incubated for 3 hours at 37° C. 5% CO2. Then, endothelial cells are harvested using Tripsine-EDTA solution (Gibco) and analyzed for caspase-3 activation by flow cytometry in living CD3⁻ cells.

### Results

CD8⁺ Tregs obtained by the method of the invention induce a low expression of active caspase 3 in endothelial cells at the highest Tcell:endothelial cell ratio.

### Example 7: Evaluation of the Cytotoxic Activity of CD8⁺ Tregs Obtained by the Method of the Invention Against Human Peripheral Blood Mononuclear Cells (PBMC)

### Material and Methods

CD8⁺ Tregs obtained by the method described in Example 5 are stained with CFSE, then added to allogeneic PBMCs in a range of Treg:PBMCs ratios from 10:1 to 1:2 in RPMI 1640 medium supplemented with 10% AB serum, Penicillin (100 U/ml), Streptomycin (0.1 mg/ml), Sodium pyruvate (1 mM), Glutamine (2 mM), Hepes Buffer (1 mM), and non-essential amino acids (lx), plates are centrifuged for 1 minute at 430 g and incubated for 18 hours at 37° C. 5% CO2. Apoptosis of monocytes, B cells, and T cells in PBMCs is analyzed by flow cytometry by gating on CD14⁺, CD19⁺, and CD3⁺ CFSE⁻ cells, respectively, using DAPI and Annexin V staining. Percent of lysis is calculated as (% lysis−% spontaneous lysis)/(% maximum lysis−% spontaneous lysis)×100.

### Results

CD8⁺ Tregs obtained by the method of the invention induce a low apoptosis in allogeneic PBMCs at the highest T cells:PBMCs ratio.

### Example 8: Transcriptomic Profile of CD8⁺ Tregs Obtained by the Method of the Invention

### Material and Methods

CD8⁺ Tregs freshly isolated from blood and cultured for 14 and 21 days in LymphoONE™, X-VIVO™ 15, ImmunoCult™-XF, PRIME-XV®, or CTS™ OpTmizer™ medium, as described in Example 5, are analyzed by bulk RNA sequencing. Genes differentially expressed between conditions are determined using the contrast function, with secondary testing (FDR) to obtain q value. Genes are considered differentially expressed if the FDR q value <0.05. Variance between samples is analyzed by principal component analysis. MA plot are drawn by plotting log 2 (fold-change) in function of the mean expression of each gene. The genes are represented on a MA plot showing the log 2 (fold-change) in function of the mean expression of each gene to highlight the underexpressed and overexpressed genes, and investigated for known function in relation with inflammation or tolerance.

### Results

The number of genes differentially expressed in CD8⁺ Tregs freshly isolated from blood and cultured for 14 and 21 days in LymphoONE™, X-VIVO™ 15, ImmunoCult™-XF, PRIME-XV®, or CTS™ OpTmizer™ medium is determined. A high number of genes differentially expressed in CD8⁺ Tregs freshly isolated from blood compared to CD8⁺ Tregs obtained by the method of the invention, with very few genes differentially expressed in CD8⁺ Tregs cultured for 14 days compared to a culture for 21 days are identified.

The genes differentially expressed in CD8⁺ Tregs freshly isolated from blood compared to CD8⁺ Tregs cultured for 14 or 21 days according to the method of the invention are related to the activation and memory status of the cells, and/or commonly associated with Tregs suppressive function.

The genes that are differentially expressed in CD8⁺ Tregs cultured for 14 days compared to a culture for 21 days are related to senescence of the cells.

### Example 9: Evaluation of the Longevity of CD8⁺ Tregs Obtained by the Method of the Invention

### Material and Methods

The CD8⁺ Tregs freshly isolated from blood and cultured for 14 and 21 days in LymphoONE™, X-VIVO™ 15, ImmunoCult™-XF, PRIME-XV®, or CTS™ OpTmizer™ medium, as described in Example 5, are lysed with the lysis buffer from Macherez-Nagel (ref: 740952), the amount of DNA is measured using a spectrophotometer, then the telomer length is determined by 40 cycles qPCR using the Absolute Human Telomere Length Quantification qPCR Assay Kit (AHTLQ)-Catalog #8918 by ScienCell.

### Results

Shorter telomers are observed in CD8⁺ Tregs cultured for 14 and 21 days in LymphoONE™, X-VIVO™ 15, ImmunoCult™-XF, PRIME-XV®, or CTS™ OpTmizer™ medium compared to CD8⁺ Tregs freshly isolated from blood. Also, shorter telomers are observed in CD8⁺ Tregs cultured for 21 days compared to CD8⁺ Tregs cultured for 14 days.

### Example 10: Genetic Modification by Lentiviral Vectors of CD8⁺ Tregs Obtained by the Method of the Invention

### Material and Methods

CD8⁺ Tregs are sorted by FACS, culture plates are coated with anti-CD3 mAb (OKT3 clone, 1 μg/ml in PBS, 100 μL/cm², 1 h at 37° C., then washed with PBS 3 times), and cells are seeded at 3×10⁵/cm²/600 μL in LymphoONE™, X-VIVO™ 15, ImmunoCult™-XF, PRIME-XV®, or CTS™ OpTmizer™ medium supplemented with IL-2 (25 U/mL, Novartis), IL-15 (10 ng/mL, Miltenyi Biotec), anti-CD28 mAb (clone CD28.2, soluble, 1 μg/ml), human serum 5% (Sigma Aldrich) and rapamycin (50 nM, Pfizer). Cells are incubated at 37° C. 5% CO₂.

At days +1 and +2, 10 MOI of lentiviral vector encoding for a transgene of about 10 kb (possibly a CAR transgene) are added or not to the culture, the plate is centrifuged for 15″ at 430 g and the cells are incubated at 37° C. 5% CO₂.

At day +3, 1 volume of in the same culture medium as used at day 0 supplemented with IL-2 (50 U/mL, Novartis), IL-15 (20 ng/mL, Miltenyi Biotec), human serum 15% (Sigma Aldrich) and rapamycin (50 nM, Pfizer) is added. Cells are incubated at 37° C. 5% CO2.

At days 7 and 14, cells are harvested, counted, washed, and seeded at 5×10⁴ Tregs/cm²/300p L in culture plate previously coated with anti-CD3 mAb (OKT3 clone, 1 μg/ml in PBS, 100 μL/cm², 1 h at 37° C., then washed with PBS 3 times), in the same culture medium as used at day 0 supplemented with IL-2 (25 U/mL, Novartis), IL-15 (10 ng/mL, Miltenyi Biotec), anti-CD28 mAb (clone CD28.2, soluble, 1 μg/ml), human serum 10% (Sigma Aldrich) and rapamycin (50 nM, Pfizer). Cells are incubated at 37° C. 5% CO₂.

Cytokines are freshly added every 2 days and fresh medium is added when required, depending on the proliferation rate.

At day 21, cells are counted and transgene expression is verified by flow cytometry.

### Results

CD8⁺ Tregs obtained by the method of the invention are transduced with high efficacy and proliferation rate of cells transduced or not are similar.

### Example 11: Genetic Modification by CRISPR-Cas of CD8⁺ Tregs Obtained by the Method of the Invention

### Material and Methods

CD8⁺ Tregs are sorted by FACS Aria, culture plates are coated with anti-CD3 mAb (OKT3 clone, 1 μg/ml in PBS, 100 μL/cm², 1 h at 37° C., then washed with PBS 3 times), and cells are seeded at 3×10⁵/cm²/600 μL in LymphoONE™, X-VIVO™ 15, ImmunoCult™-XF, PRIME-XV®, or CTS™ OpTmizer™ medium supplemented with IL-2 (25 U/mL, Novartis), IL-15 (10 ng/mL, Miltenyi Biotec), anti-CD28 mAb (clone CD28.2, soluble, 1 μg/ml), human serum 10% (Sigma Aldrich) and rapamycin (50 nM, Pfizer). Cells are incubated at 37° C. 5% CO₂.

At day +2, cells are electroporated or not using the Neon (ThermoFisher) or Amaxa (Lonza) with CRISPR-Cas9 complexes (targeting B2M for example) and the cells are incubated at 37° C. 5% CO2.

At days +7 and +14, cells are harvested, washed, and seeded at 5×10⁴ Tregs/cm²/300p L in culture plate previously coated with anti-CD3 mAb (OKT3 clone, 1 μg/ml in PBS, 100 μL/cm², 1 h at 37° C., then washed with PBS 3 times), in the same culture medium as used at day 0 supplemented with IL-2 (25 U/mL, Novartis), IL-15 (10 ng/mL, Miltenyi Biotec), anti-CD28 mAb (clone CD28.2, soluble, 1 μg/ml), human serum 10% (Sigma Aldrich) and rapamycin (50 nM, Pfizer). Cells are incubated at 37° C. 5% CO₂.

Cytokines are freshly added every 2 days and fresh medium is added when required, depending on the proliferation rate.

At day 21, cells are counted and knockout efficacy is assessed by flow cytometry.

### Results

CD8⁺ Tregs obtained by the method of the invention are electroporated and knocked-out for the targeted gene with high efficacy and proliferation rate of cells electroporated or not are similar.

1-15. (canceled)

16. An in vitro method for obtaining clinical-grade CD8⁺ CD45RC^{low/-} regulatory T (Treg) cells, comprising the steps of:
(a) isolating CD8⁺ cells from a biological sample containing human peripheral blood mononuclear cells (PBMCs) or lymphocytes,
(b) isolating CD8⁺ CD45RC^{low/-} Treg cells from the cells obtained at step (a) by flow cytometry in a closed system using a clinical-grade anti-CD45RC antibody,
(c) stimulating CD8⁺ CD45RC^{low/-} Treg cells obtained at step (b) with a clinical-grade anti-CD3 antibody and a clinical-grade anti-CD28 antibody,
(d) culturing the stimulated Treg cells obtained at step (c) in a medium selected from the group consisting of X-VIVO™ 15, LymphoONE™, ImmunoCult™-XF, PRIME-XV® and CTS™ OpTmizer™, said medium being supplemented with clinical-grade rapamycin and clinical-grade IL-2.

17. The in vitro method according to claim 16, wherein step (a) is performed with a clinical-grade anti-CD8 antibody.

18. The in vitro method according to claim 17, wherein step (a) is performed with a clinical-grade anti-CD8 antibody coated to beads.

19. The in vitro method according to claim 16, wherein step (b) comprises isolating CD8⁺ CD45RC^{low/-} CD56⁻ Treg cells by further using an anti-CD56 antibody.

20. The in vitro method according to claim 16, wherein step (a) is performed using CliniMACS® Plus System (Miltenyi Biotec) and/or step (b) is performed using a cell sorter selected from the group consisting of MACSQuant® Tyto® Cell Sorter, CGX10 (Sony) and WOLF® Cell Sorter.

21. The in vitro method according to claim 16, wherein the anti-CD3 antibody and the anti-CD28 antibody are both used at a concentration of between 1 μg/mL and 10 μg/mL.

22. The in vitro method according to claim 21, wherein the IL-2 concentration at step (d) is of between 25 U/mL and 1000 U/mL; and/or wherein the rapamycin concentration at step (d) is of between 25 nM and 100 nM.

23. The in vitro method according to claim 16, wherein the medium of step (d) is supplemented with a serum selected from the group consisting of CTS™ Immune cell serum replacement, and human serum, and/or wherein the medium of step (d) is supplemented with IL-15.

24. The in vitro method according to claim 16, wherein, at step (d), the cells are seeded at at least 1×10⁵ cells/cm² into the recipient of culture at day 0.

25. The in vitro method according to claim 16, wherein the duration of step (d) is of between 14 days and 31 days.

26. The in vitro method according to claim 16, wherein the duration of step (d) is of 21 days.

27. The in vitro method according to claim 16, further comprising:
(e) stimulating the cells obtained at step (d) with a clinical-grade anti-CD3 antibody and a clinical-grade anti-CD28 antibody.

28. The in vitro method according to claim 27, wherein step (e) is performed after 7 and/or 14 days of culture.

29. The in vitro method according to claim 16, wherein steps (a) to (d) are performed in closed systems.

30. The in vitro method according to claim 27, wherein steps (a) to (e) are performed in closed systems.

31. The in vitro method according to claim 16, wherein the clinical-grade CD8⁺ CD45RC^{low/-} regulatory T (Treg) cells obtained by said method are GITR+, CD127− and/or Foxp3+ cells.

32. A pharmaceutical composition comprising clinical-grade CD8⁺ CD45RC^{low/-} regulatory T (Treg) cells obtainable by the in vitro method according to claim 16.

33. A method of treating a subject in need thereof, said method comprising administering to said subject a pharmaceutical composition comprising clinical-grade CD8⁺ CD45RC^{low/-} regulatory T (Treg) cells obtainable by the in vitro method according to claim 16.

34. A method of preventing or treating transplant rejection, graft-versus-host-disease (GVHD), autoimmune disease, chronic inflammatory disease or allergy in a subject in need thereof, said method comprising administering to said subject a pharmaceutical composition comprising clinical-grade CD8⁺ CD45RC^{low/-} regulatory T (Treg) cells obtainable by the in vitro method according to claim 16.
